(19) United States
(12) Patent Application Publication
(10) Pub. No.: US 2026/0297536 A1
(43) Pub. Date: Oct. 1, 2026

(54) SYSTEM, APPARATUS AND METHOD FOR SPHEROIDAL PLURIPOTENT STEM CELL GENERATION USING COLD-TRIGGERED DETACHMENT

(71) Applicant: HOHCells, LLC, College Park, MD (US)

(72) Inventor: Xiaoming He, Bethesda, MD (US)

(21) Appl. No.: 19/090,572

(22) Filed: Mar. 26, 2025

(51) Int. Cl. C12N 5/074

(52) U.S. Cl. CPC C12N 5/0696; C12N 2509/00; C12N 2513/00; C12N 2533/30; C12N 2533/90; C12N 2535/10

(57) ABSTRACT

A cold-responsive micropatterned dish (crMPD) is developed by spin-coating a thin layer of cold-responsive polymer on cell-culture dish and further microcontact printing cell attachment micropatterns on top of the coating layer. The hiPSCs attach and proliferate exclusively within the micropatterned areas to form a large number of uniform hiPSC colonies that can be detached as a whole simply by putting the crMPD on ice for ~5-15 minutes. Under 3D culture without RI, the colonies can quickly self-assemble into homogeneous hiPSC spheroids with high viability, yield, and pluripotency. This crMPD technology may be invaluable to facilitate widespread application of hiPSCs in research and personalized medicine.

### FIELD OF THE INVENTION

The technology of the instant invention concerns an improved technique to prepare spheroidal stem cells, particularly ways to improve the yield of pluripotent stem cells for the multitude of medical uses now and to come. The invention also involves improvements in thermoresponsive materials, particularly cold-responsive materials, used for dissociation or detachment mechanisms, particularly involving cold-triggered detachments, enabling the production of stem cells without the usage of deleterious inhibitors.

### BACKGROUND OF INVENTION

Human induced pluripotent stem cells (iPSCs) show great promise for personalized cell-based medicine, as they can be derived from easily accessible somatic cells and differentiated into all three germ layers without ethical concerns. This requires mass production of human iPSC (hiPSC) in 3D. However, contemporary methods for 3D culture result in hiPSC spheroids with significant size heterogeneity that is undesired for controlled differentiation and require the use of high concentration of Rho-associated kinase (ROCK) inhibitor (RI) to improve the cell viability. Unfortunately, high concentration of RI causes uncontrolled spontaneous differentiation, necessitating a new approach that detaches the cultivated hiPSC spheroids without the harmful inhibitors.

Human induced pluripotent stem cells (hiPSCs), with their capability of differentiating into cell types of all three germ layers, hold tremendous potential for personalized cell-based medicine. The use of autologous hiPSCs compared to human embryonic stem cells (hESCs, usually not autologous) avoids concerns over immunoreactions and ethical issues. The hiPSCs have numerous applications for personalized modeling and treating disease, such as cell-based therapy, tissue and organ engineering, drug discovery, and toxicological screening. For many of these applications, large-scale production of the hiPSCs via 3D culture to form hiPSC spheroids, is indispensable. These 3D hiPSC spheroids mimic some structure of the developing embryo and are used to start lineage-specific differentiation towards many different lineages like cardiac and neural. Cell differentiation outcomes using hiPSCs spheroids depend greatly on the homogeneity and quality of the hiPSC spheroids. The size of the pluripotent cell spheroids plays a large role in the effectiveness of differentiation to a specific lineage, with spheroids that vary too greatly in size compromising the purity of directed differentiation. Spheroids of controllable and reproducible size are ideal for the best cell quality, viability, and directed differentiation into specific lineages.

Several methodologies have been developed with the goal of mass-producing reliable and reproducible pluripotent spheroids, such as hanging drop/suspension culture, microwell arrays, microfluidics, and bioreactors. Although promising, these methods are oftentimes time-consuming, complex, labor-intensive, and/or expensive. Two-dimensional (2D) culture systems offer several advantages for hiPSC culture, namely for pluripotency maintenance and quality control. To create multicellular spheroids from 2D culture systems, hiPSC colonies must be detached from the 2D surface and allowed to self-assemble into spheroids. Conventional detachment methods using enzymatic or chelating agents, such as the commercially available Versene, more often result in dissociation of the hiPSCs into single cells, which may quickly die of apoptosis and thereby result in poor spheroid yield[9a]. Protective agents like Rho-associated kinase (ROCK) inhibitor (RI), Y-27632, can be added to suppress dissociation-induced apoptosis of hiPSCs in suspension culture.

However, adding RI is not ideal for hiPSC spheroid production for cell-based medicine as adding a xeno-product introduces the risk of adverse effects or toxicities. Furthermore, RI has been shown to decrease pluripotency markers in 3D hiPSC spheroids and subsequently negatively impact their directed cardiac differentiation. Other methods force cell aggregation via centrifugation, which can potentially damage the cells and adds more steps to 3D hiPSC spheroid protocols. Therefore, there is a need for better protocols to produce 3D hiPSC spheroids that are highly pluripotent and homogenous in size, and do not require the use of RI.

Recently, there has been considerable interest in developing cold-responsive cell culture surfaces as a method for enzyme-free cell detachment. One of the most common polymers used for this application is poly(N-isopropyl acrylamide-butyl acrylate) (pNIPAAm). This cold-responsive polymer has the unique property of undergoing a phase/conformation transition when cooled below its lower critical solution temperature (LCST), altering the property of the polymer from a hydrophobic nature to hydrophilic one. Reducing the temperature of the cell culture dish can thereby facilitate cell detachment without altering cell morphology or quality. However, many of the pNIPAAm co-polymers used have cell detachment temperatures of ~16-30 °C, which can make cell culture difficult, as they are close to room temperature (~22 °C) and cell culture involves operations (e.g., changing medium and observing cells under microscope) at room temperature.

Additionally, most studies evenly coat an extracellular matrix (ECM) component on top of the pNIPAAm copolymer for cell attachment, to produce large cell sheets or detaching hiPSC colonies of uncontrolled sizes. Microcontact printing, or micropatterning, is a simple technique to precisely and gently transfer a bioink like a protein solution from a stamp design to a surface. Although this technique has been used to design cell attachment domains of uniform in size to produce similarly sized 2D hiPSC and hESC colonies, it has never been used to fabricate ink patterns on an ice cold-responsive surface.

In this study, a pNIPAAm-butyl acrylate (BA) (pNIPAM-BA or pNIPAm-BA) copolymer is synthesized to have an LCST of about 0-12 °C, about 2-8 °C or about 4-8 °C for surface coating to make a cold-responsive surface for convenient ice cold-triggered cell detachment. This cold-responsive surface is then micropatterned with a design of Matrigel islands with controllable and uniform size to produce homogeneous hiPSC colonies. This cold-responsive micropatterned dish (crMPD) can then be cooled on ice (~0 °C) to gently release the homogeneous hiPSC colonies as a whole with negligible single hiPSCs in the absence of any enzymatic or chelating detachment agents. As a result, these hiPSCs are able to form hiPSC spheroids with high viability, high yield, high homogeneity, and high expression of pluripotency markers in the absence of RI. This novel crMPD-based method is not only cost-and time-effective compared to other hiPSC spheroid production methods, but also produce high-quality hiPSC spheroids with a homogeneous size, which are desirable characteristics for downstream differentiation of the hiPSCs into specific cell lineages, to facilitate the widespread use of hiPSCs for fundamental research and personalized cell-based medicine.

### SUMMARY OF THE PRESENT INVENTION

To address these grand challenges using these techniques, a cold-responsive micropatterned dish (crMPD) is thus developed by spin-coating a thin layer of cold-responsive polymer on cell-culture dish and further microcontact printing cell attachment micropatterns on top of the coating layer. The hiPSCs attach and proliferate exclusively within the micropatterned areas to form a large number of uniform hiPSC colonies that can be detached as a whole simply by putting the crMPD on ice for ~5-15 minutes. Under 3D culture without RI, the colonies can quickly self-assemble into homogeneous hiPSC spheroids with high viability, yield, and pluripotency. This ingenious crMPD technology may be invaluable to facilitate widespread application of hiPSCs in research and personalized medicine.

### BRIEF DESCRIPTION OF THE DRAWINGS

While this Specification concludes with claims particularly pointing out embodiments and distinctly claiming the subject matter that is regarded as forming the present invention, it is believed that the invention will be better understood from the following Description taken in conjunction with the accompanying Drawings, where like reference numerals designate like system signal flow and other mechanical elements, in which:

FIGS. 1A and 1B illustrate an embodiment of a process for making spheroidal stem cells using a cold-responsive micropatterned technique pursuant to the principles of the present invention;

FIGS. 2A-2D illustrate, using scanning electron microscopy images and graphical analyses, uncoated and cold-responsive layer coated surfaces;

FIGS. 3A and 3B further illustrate comparisons between uncoated and cold-responsive layer coated surfaces;

FIG. 4 illustrates contact angle analyses of water droplets on uncoated and pNIPAAm-BA coated surfaces pursuant to the teachings of the instant invention;

FIGS. 5A-5C illustrate micropatterned designs, cultures and cold-triggered release, along with analyses, pursuant to an embodiment of the instant invention;

FIG. 6 are fluorescence images of micro-contact and patterning using the techniques of the present invention;

FIGS. 7A and 7B illustrates detachment viability and yield of the spheroidal stem cells from the layer, along with graphical analyses, according to an embodiment of the present invention;

FIG. 8 further illustrate detachment viability and yield of the spheroidal stem cells;

FIG. 9 illustrates quantitative viability data and analyses for the detachment of the spheroidal stem cells from the layer, as shown in FIGS. 7 and 8;

FIG. 10 further illustrates additional detachment viability and yield of reprogrammed spheroidal stem cells from the layer using stain, pursuant to the teachings of the present invention;

FIG. 11 illustrates analyses for the detachment viability and yield of the spheroidal stem cells from the layer, as shown in FIG. 10;

FIG. 12 illustrates additional analyses for the detachment viability and yield of the spheroidal stem cells from the layer pursuant to the present invention;

FIG. 13 further illustrates additional detachment viability and yield of reprogrammed spheroidal stem cells from the layer using stain, pursuant to the teachings of the present invention;

FIG. 14 illustrates average size dimensions of the spheroidal stem cells in three embodiments generated pursuant to the teachings of the present invention;

FIG. 15 shows exemplary homogenous size stem cells produced from the techniques of the present invention, along with analyses of the same;

FIG. 16 further shows exemplary homogenous size stem cells produced from the techniques of the present invention, along with analyses of the same;

FIG. 17 further illustrates and analyzes reprogrammed spheroidals produced pursuant to the teachings of the present invention;

FIGS. 18A-18E illustrate and analyze the pluripotency of the stem cells produced by the techniques of the present invention;

FIG. 19 are representative flow cytometry peaks showing the expression of pluripotency markers in hiPSC spheroids produced by Versene detachment; and

FIG. 20 is a graphic generally illustrating the maturation and detachment of the stem cells on the cold-responsive micropatterned surface pursuant to teachings and illustrated embodiments of the present invention.

### DETAILED DESCRIPTION OF THE PRESENT INVENTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications provided herein are only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

As discussed, there is a present need for improved techniques to produce stem cells in massive quantities. Prior art techniques employ inhibitors in the process, particularly involving detachment of the stem cells from a substrate. Since these inhibitors themselves affect the integrity of the spheroidal stem cell process, an improved technique that avoids usage of these deleterious chemicals is a huge step forward.

The technique employed in the present invention is an elegant approach, depositing a micropattern of cell attachment sites onto a thermo-responsive or cold-responsive layer within a dish or device, where the layer has the property where, when cooled below a critical temperature, the chemical properties change, enabling detachment of the grown stem cells without the use of inhibitors.

Various studies of this phenomenon and new approaches are set forth hereinbelow to further elucidate the properties and processes of the instant invention.

Fabrication and Characterization of the crMPD

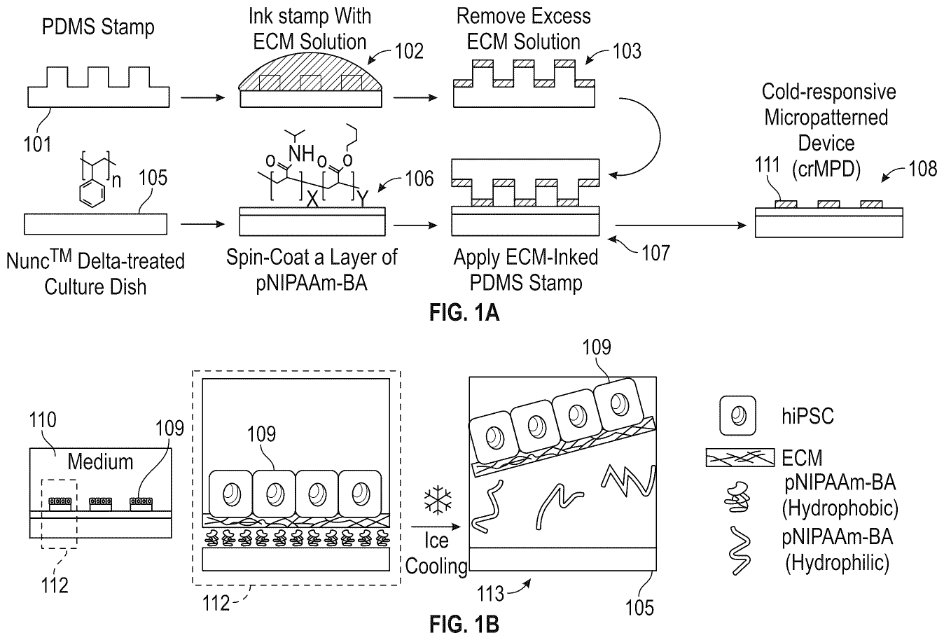

This study demonstrates the fabrication, as shown in FIG. 1A, and use, as shown in FIG. 1B, of a cold-responsive micropatterned dish (crMPD) for convenient mass production of human induced pluripotent and homogenous stems cell (hiPSC) spheroids with high viability and pluripotency without the use of any Rho-associated kinase (ROCK) inhibitor (RI) during 3D culture.

With reference now to FIGS. 1A and 1B of the DRAWINGS, there are shown schematic illustrations of the fabrication and use of the cold-responsive micropatterned device (crMPD). As shown in FIG. 1A, a PDMS stamp, generally designated by the reference numeral 101, pre-fabricated with the desired features for surface-patterning with cell attachment areas (ECM) is inked with the ECM solution. Then, the excess ECM solution is removed via aspiration and blowing with N₂. Concurrently, a Nunc™ Delta-treated polystyrene dish is spin-coated or spray-coated with a layer of cold-responsive polymer, pNIPAAm-BA (x:y=5.8, x and y are the molar amount of NIPAAm and BA, respectively), to create a cold-responsive surface.

As shown, the inked PDMS stamp 101 is then placed pattern side down onto the cold-responsive surface to pattern the surface with ECM for cell attachment, resulting in the final product, the crMPD. The ECM pattern used in this study is circular islands of Matrigel for hiPSC attachment. As shown in FIG. 1B, hiPSCs can attach on the crMPD only where there are the Matrigel islands created by the micropatterning with the PDMS stamp. At about 37 °C, the pNIPAAm-BA is insoluble in water/medium, and the polymer coating on the dish remains intact. Upon ice cooling (~0 °C) below the LCST of the polymer (4-8 °C), the pNIPAAm-BA becomes more hydrophilic and dissolves in the culture medium, releasing the hiPSCs as a whole cluster/colony from the surface.

To create the crMPD, as shown in FIG. 1A, stamp 101, made of a polydimethylsiloxane (PDMS), is first made via photolithography and soft lithography with the desired micropatterns or islands. This stamp 101 is inked with an extracellular matrix (ECM) solution (such as 20 μg/mL Matrigel) to allow the proteins for cell attachment to adsorb onto the micropatterns of the PDMS stamp, generally designated by the reference numeral 102, and the excess ECM solution is then removed, generally designated by the reference numeral 103. Simultaneously, a Nunc™ Cell Culture/Petri Dishes (product #: 153066, ThermoFisher Scientific) or Nunc™ Delta-treated polystyrene dish, generally designated by the reference numeral 105, is spin-coated or spray-coated with a cold-responsive polymer, such as poly(N-isopropyl acrylamide-butyl acrylate) or pNIPAAm-BA, generally designated by the reference numeral 106.

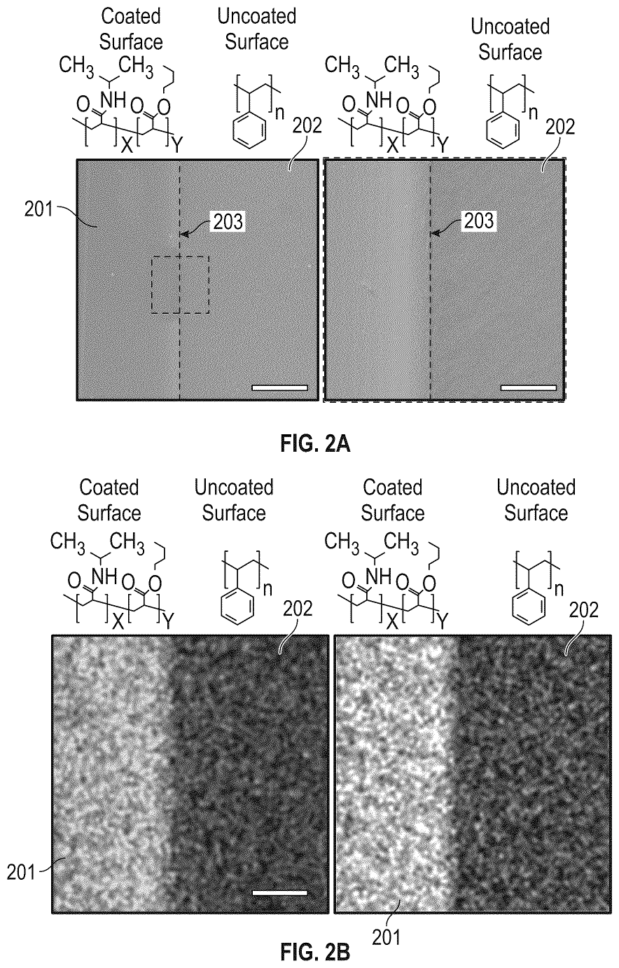

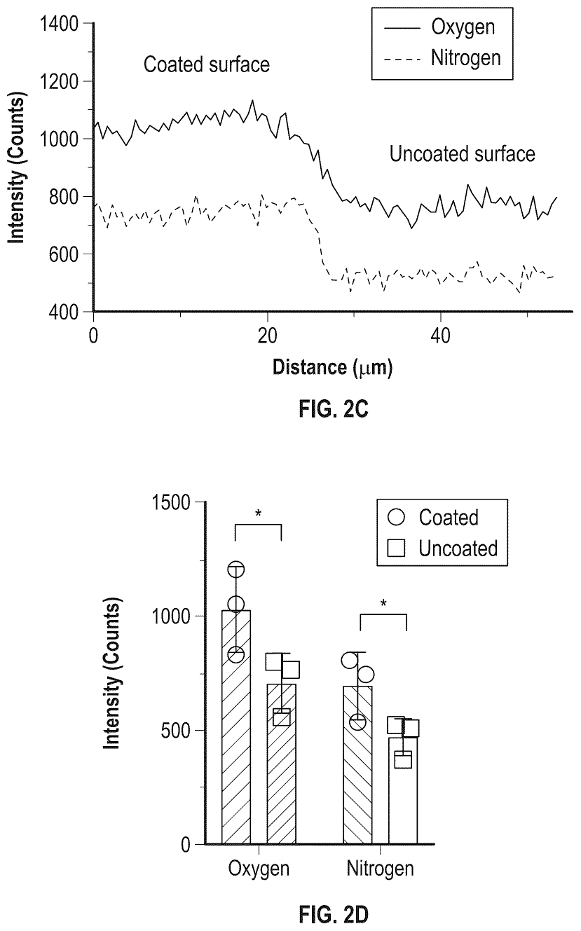

With reference now to FIG. 2A, there are shown SEM and EDXS analyses of uncoated surface and pNIPAAm-BA coated surface. Representative Scanning electron microscopy (SEM) image (left) together with a zoom-in view (right) showing the interface of the uncoated Nunc™ Delta-treated polystyrene surface and pNIPAAm-BA coated surface. Scale bars: 50 μm (left) and 10 μm (right). With reference now to FIG. 2B, energy dispersive X-ray spectroscopy (EDXS) mapping of oxygen (left) and nitrogen (right) in the surface layer of the uncoated versus coated surface is shown. The brightness represents the intensity of the element's signal. The weak signals of nitrogen and oxygen on the uncoated side are due to residual oxygen and nitrogen gases in the sample chamber and the Nunc™ Delta treatment. Scale bar: 10 μm. With reference now to FIG. 2C, there is shown an EDXS line scan from the coated to uncoated surface. The intensity of both oxygen and nitrogen on the coated surface is higher than that on the uncoated surface. With reference to FIG. 2D, there is shown a comparison of the average intensity of oxygen and nitrogen from the EDXS line scans of the coated surface versus uncoated surface. The x:y ratio of pNIPAAm-BA copolymers is 5.8. Statistical analyses were done using two-tailed paired t-test. *p<0.05. n=3 independent runs. Error bars represent standard deviation.

Successful coating of pNIPAAm-BA 106 on the surface of the polystyrenes cell culture dish 105 is confirmed by scanning electron microscopy (SEM) image, as shown in FIG. 2A, of the area with an interface, generally designated by the reference numeral 203, between the uncoated, generally designated by the reference numeral 202, and the pNIPAAm-BA coated surface, generally designated by the reference numeral 201. The successful coating is further confirmed by energy dispersive X-ray spectroscopy (EDXS) analysis of nitrogen (N) and oxygen (O) elements in the coated versus uncoated surface layers.

As shown in FIGS. 2B, 2C and 2D, the pNIPAAm-BA coated surface 201 has significantly higher occurrence of both N and O elements than the uncoated surface, because the pNIPAAm-BA polymer in the coated surface 201 contains both N and O while the polystyrene polymer in the uncoated surface does not. The weak signals of nitrogen and oxygen on the uncoated side are due to residual oxygen and nitrogen gases in the sample chamber of EDXS and the aforementioned Nunc™ Delta treatment.

After then excess ECM solution 103 is aspirated and blown off the PDMS stamp 101 with nitrogen gas, the inked stamp is placed feature side down on the pNIPAAm-BA coated surface 106/201, to microcontact print the cold-responsive surface with ECM micropatterns, generally designated by the reference numeral 107, where the cells can then attach, creating the crMPD, generally designated by the reference numeral 108.

As shown in FIG. 1B, cells, generally designated by the reference numeral 109, within a medium, generally designated by the reference numeral 110, can then be seeded onto the crMPD 108, where the cells 109 attach only on the ECM pattern, generally designated by the reference numeral 111, as shown regularly displaced in FIG. 1B. A grouping of cells 109 for one of the ECM pattern structures 111 is also shown, generally designated by the reference numeral 112.

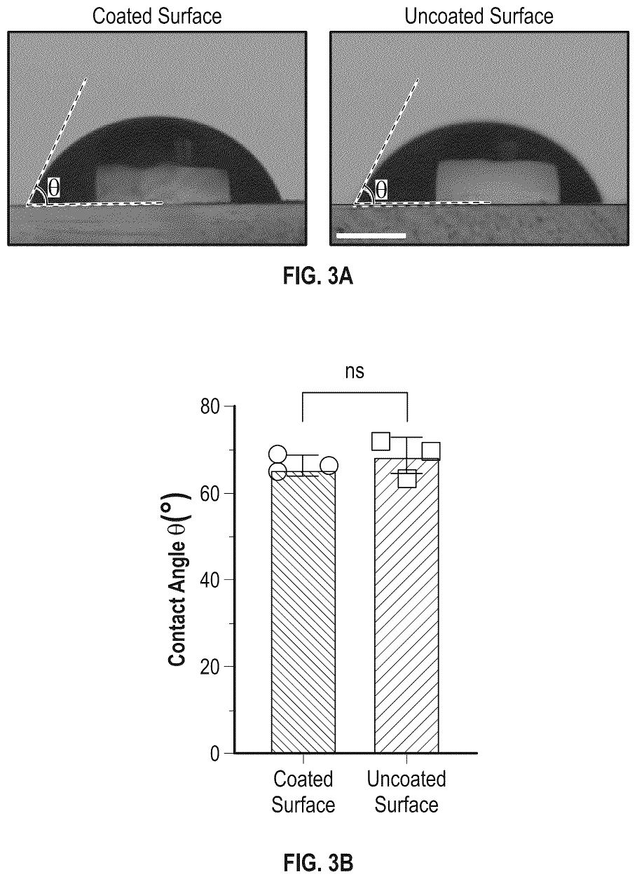

With reference now to FIG. 3A, there are shown contact angle analyses of water droplets on uncoated and pNIPAAm-BA coated surfaces. Representative images of the water droplet on uncoated Nunc™ Delta-treated polystyrene surface (left) and pNIPAAm-BA (x:y=5.8, x for NIPAAm and y for BA) coated surface (right) showing the contact angle (θ). With reference now to FIG. 3B, quantitative data of contact angles of water droplets on the uncoated versus pNIPAAm-BA coated surface are shown. Statistical analyses were done using two-tailed unpaired t-test. ns represents not significant. n=3 independent runs. Error bars represent standard deviation. Scale bar: 100 μm.

This seeding is because the hydrophilicity (or hydrophobicity) of the coated surface 106/201 is not significantly different from the aforementioned uncoated Nunc™ Delta-treated polystyrene surface 202, according to a contact angle analysis, such as shown in FIGS. 3A and 3B. It is worth noting that the contact angle of the uncoated Nunc™ Delta-treated surface (~65°), generally designated by the reference numeral 302, is smaller than the contact angle of the known untreated polystyrene surface (~90°), generally designated by the reference numeral 301, indicating the Nunc™ Delta treatment makes the polystyrene surface more hydrophilic.

To release the cells from the ECM micropatterns 111 in the crMPD 108, the dish 105 is placed on ice (~0 °C) to cool the pNIPAAm-BA coating 106 below its lower critical solution temperature (LCST), which is about 0-12 °C, about 2-8 °C or about 4-8 °C. As the pNIPAAm-BA coating 106 is cooled below its LCST, generally designated by the reference numeral 113, the polymer undergoes a phase change to become hydrophilic and water soluble, whereby it dissolves in the medium 110 and releases the cell 109 colony, as a whole, from the dish 105 without the need of any enzyme or chelating agent to detach them from the surface, as illustrated in FIG. 1B.

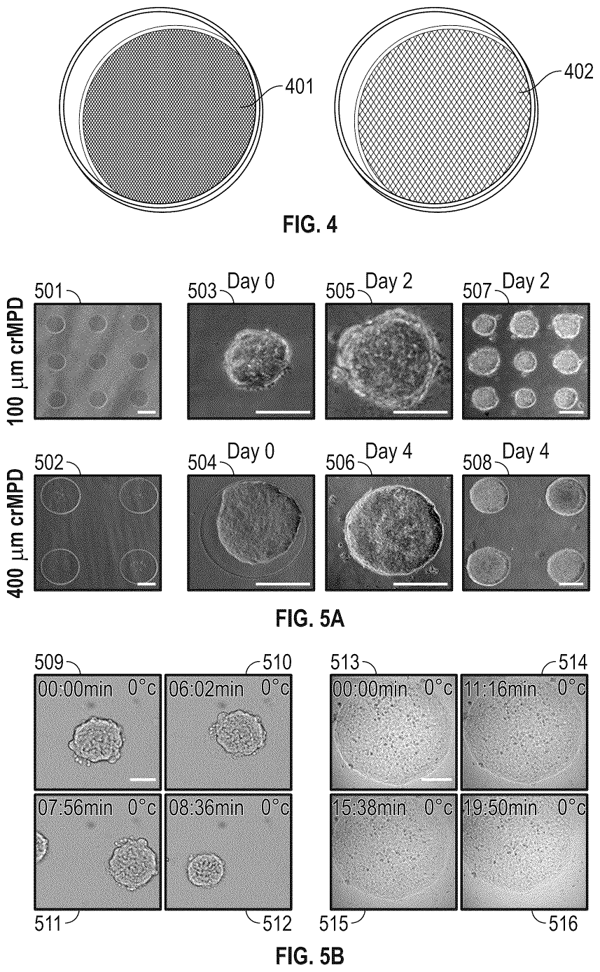

Wit reference now to FIG. 4 of the DRAWINGS, there are shown representative photos of crMPDs with Matrigel micropatterns of different sizes. In one image, to the left, 100 μm micropatterns are shown. In the right image, 400 μm micropatterns are shown. The micropatterns can be seen as the many tiny white dots in the photos. Approximately 2,000 micropatterns of 400 μm and 15,000 micropatterns of 100 μm can be made in a 35 mm dish. Scale bars: 1 mm.

To demonstrate successful fabrication of cell attachment micropatterns on the pNIPAAm-BA coated surface of the crMPD, dishes 105 with Matrigel islands, generally designated by the reference numerals 401 and 402, respectively, of both 100 μm and 400 μm (in diameter) on the crMPD 108 are fabricated, as illustrated in FIG. 4.

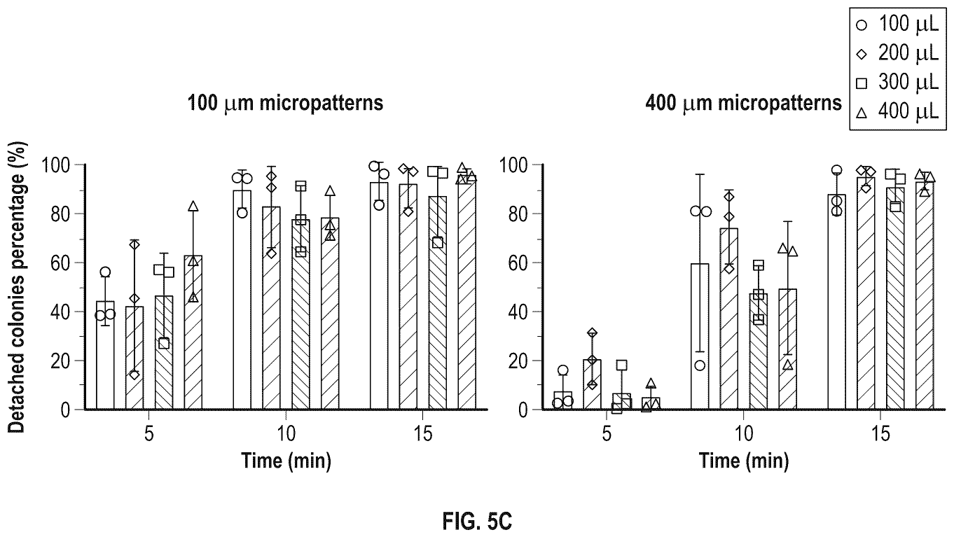

With reference now to FIGS. 5A-5C of the DRAWINGS, there is illustrated crMPD micropattern design and culture and cold-triggered release of hiPSCs in the crMPD. Phase images of the micropatterned ECM (Matrigel) islands of different sizes (100 μm and 400 μm) on the cold-responsive pNIPAM-BA layer in the crMPD.

Both 100 and 400 μm Matrigel islands are uniform and round. Scale bars: 100 μm for panel A and 200 μm for panel B. C-D, DF19-9-11T.H hiPSC attachment on 100 μm (C) and 400 μm (D) Matrigel islands/micropatterns on crMPD. The hiPSCs could attach and grow in 2-4 days to form well defined colonies with clear boundary on both 100 μm and 400 μm Matrigel islands. Scale bars: 100 μm for panel C and 200 μm for panel D.

With reference to FIG. 5B, there are shown cryomicroscopy images showing detachment of Matrigel islands of different size (100 μm, and 400 μm) from the crMPD upon cooling at 0 °C. Scale bars: 50 μm for panel E and 100 μm for panel F. With reference to FIG. 5G, there is shown detaching percentage of hiPSC colonies grown in crMPDs with different coating thicknesses of pNIPAM-BA at different time points of cooling at 0 °C. The 100, 200, 300, and 400 μL of pNIPAAm-BA solution represent 5.7, 11.4, 17.0, and 22.7 μm of coating thickness. There is no statistical difference in the detaching percentage between the four different pNIPAM-BA coating thicknesses for both the 100 μm (left) and 400 μm (right) micropatterns at same detaching time points. Good detachment percentage (~90%) can be achieved for micropatterns of both sizes after cooling on ice for 15 mins. Statistical analyses were done using one-way analysis of variance (ANOVA) with Tukey's multiple comparisons test and correction. n=3 independent runs. Error bars represent standard deviation.

With additional reference to FIG. 5A, microscopic phase images of the dishes 105 confirm that the Matrigel islands 401 and 402 are successfully patterned onto the pNIPAAm-BA surface 106 in the dishes 105 with good homogeneity at the designed size (~100 μm in diameter), generally designated by the reference numeral 501, and ~400 μm in diameter, generally designated by the reference numeral 502. To confirm the homogeneity of ECM materials inside each island 401 and 402, a mixture of fluorescent Rhodamine B and Matrigel was used as the ECM inking solution 102 for making the micropatterns 111.

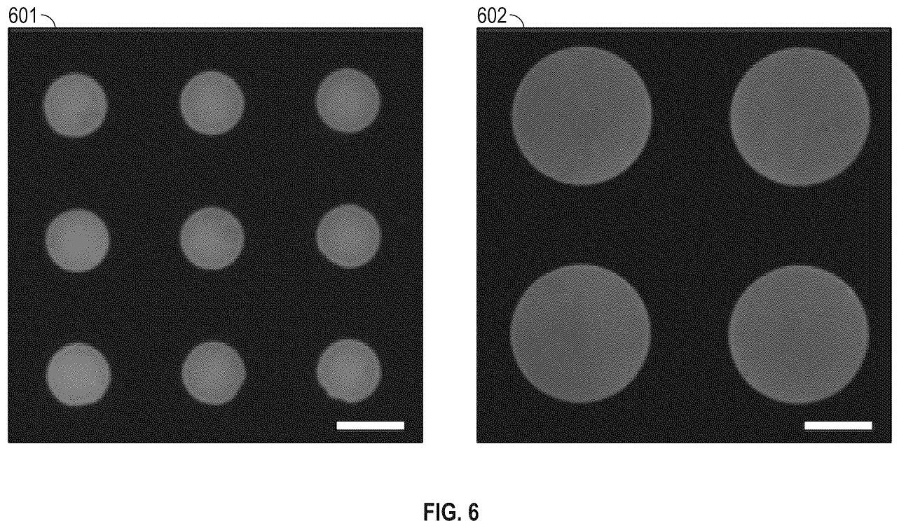

With reference now to FIG. 6 of the DRAWINGS, there are shown fluorescence images 601 and 602 of the micropatterns made with a mixture of Matrigel and Rhodamine B as the ink. In part A on the left, 100 μm micropatterns are shown. In part B on the right, 400 μm micropatterns are illustrated. The fluorescence images show uniform distribution of Rhodamine B (with red fluorescence) in the micropatterns. Scale bars: 100 μm for panel A and 200 μm for panel B.

The crMPD for hiPSC Culture and Cold-Triggered Release

To confirm that hiPSCs can attach, grow/proliferate, and then subsequently detach from the crMPD, DF19-9-11T.H cells were seeded on a crMPD with micropatterned Matrigel islands of 100 μm and 400 μm in diameter at a density of 1×104 cells/cm². With further reference to FIG. 5A, after culturing overnight, hiPSCs can be seen to successfully attach at Day 0, generally designated by the reference numerals 502 and 503, and proliferate (i.e., the colony becomes bigger).

As shown in FIG. 5A, at the start, at Day 0, at Day 2 (high magnification), and at Day 2 (low magnification) for 100 μm patterns, generally designated by the reference numerals 501, 503, 505 and 507, respectively, and at the start, at Day 0, at Day 4 (high magnification), and at Day 4 (low magnification) for 400 μm patterns, generally designated by the reference numerals 502, 504, 506 and 508, respectively, on the micropatterned Matrigel islands in the crMPD. The hiPSC growth and proliferation are confined only to the Matrigel islands micropatterned on the pNIPAAm-BA, allowing for the creation of defined hiPSC patterns, which in this case are round colonies of hiPSCs as seen in the lower magnification image of the crMPD on Day 2 for 100 μm patterns, generally designated by the reference numeral 505 and Day 4 for 400 μm patterns, generally designated by the reference numeral 508. After 2 or 4 days of culture in the crMPD, hiPSCs maintain normal colony morphology (compact/tight cellular packing with defined and smooth colony boundaries) on the Matrigel coated areas of the crMPD.

Next, cryomicroscopy was used to confirm that cooling below the LCST of pNIPAAm-BA can result in gentle and easy detachment of the 2D hiPSC colonies from the crMPD. The DF19-9-11T.H hiPSCs were seeded in a crMPD patterned with round Matrigel island of 100 μm in diameter and cultured for 2 days, or 400 μm in diameter and cultured for 4 days, and then cooled on a cryomicroscope stage to about 0 °C. The temperature of the sample was held at about 0 °C and images of the hiPSC colony grown on the round island attachment site were taken over either 10 min (for 100 μm Matrigel islands) or 20 min (for 400 μm Matrigel islands).

As shown in FIG. 5B of the DRAWINGS, to the left, for the 100 μm micropatterns, the round ~100 μm hiPSC 2D colony can be observed to attach on the Matrigel island at 00:00 minutes, generally designated by the reference numeral 509. After ~6 min of cooling at 0 °C, the hiPSC colony begins to detach from the round island, as it moves slightly away to the top and right from its original position in the image generally designated by the reference numerals 510 and 511. The hiPSC colony continues to detach or move away from its original position in the crMPD until it leaves the imaging region completely by 08:36 min, generally designated by the reference numeral 512, when another hiPSC colony that has detached from a different round island attachment site enters the imaging region.

As also shown in FIG. 5B, to the right, for the 400 μm micropatterns, the round ~400 μm hiPSC 2D colony can be observed to attach on the Matrigel island at 00:00 minutes, generally designated by the reference numeral 513. After ~11 min of cooling at 0 °C, the hiPSC colony begins to detach from the round island, generally designated by the reference numeral 514 and 515, as it moves slightly away to the top and left from its original position in the image. The 400 μm hiPSC colony continues to detach or move away from its original position in the crMPD until ~20:00 at the end of video recording, generally designated by the reference numeral 516. Compared to the 100 μm hiPSC colony, it exhibits smaller movements due to its much (~4 times in diameter and 16 times in area) larger size and weight.

To further assess the impact of pNIPAM-BA coating thickness on detachment efficiency, four different volumes of pNIPAM-BA (in ethanol) solutions were used to spin coat pNIPAM-BA on the 35 mm cell culture dish, resulting in crMPDs with four different thickness of the pNIPAM-BA coating layer. Both 100 μm and 400 μm (diameter) micropatterns were microcontact printed on the resultant crMPDs. Then, DF19-9-11T.H hiPSCs were seeded and cultured in the crMPDs for either 2 days (100 μm Matrigel islands) or 4 days (400 μm Matrigel islands). The percentage of detached hiPSC colonies from the crMPDs coated with different pNIPAM-BA thicknesses was then quantified after 5, 10, and 15 minutes of cooling on ice, and the results are shown in FIG. 5C. There is no statistical difference between the different coating thickness of pNIPAM-BA for both 100 μm, the graph at the left generally designated by the reference numeral 517, and 400 μm, the graph at the right designated by the reference numeral 518, crMPDs at all time points. Both the 100 μm calculations and the 400 μm calculations for crMPD show good (~90%) detachment percentages after cooling on ice for 15 minutes, although the detachment percentage appears higher for the smaller micropatterns at 10 min.

High Viability and Yield of hiPSCs Cold-Detached From crMPD in 3D Culture Without RI

The viability of hiPSCs detached from the crMPD with 400 μm (in diameter) Matrigel islands was also investigated using a live/dead assay assessed by calcein AM (live/green) and propidium iodide (PI, dead/red) staining with three different hiPSC cell lines: DF19-9-11T.H and IMR90-1 purchased from WiCell, and a home-made iPSC cell line reprogrammed from HPF using a commercial reprogramming kit. The hiPSCs were detached using either Versene or cold via the crMPD, then cultured in 3D for either 2 or 48 hours before staining. The hiPSCs detached using Versene were supplemented with 0, 1, or 10 μM RI for 3D culture, while the hiPSCs detached from the crMPD were not supplemented with any RI.

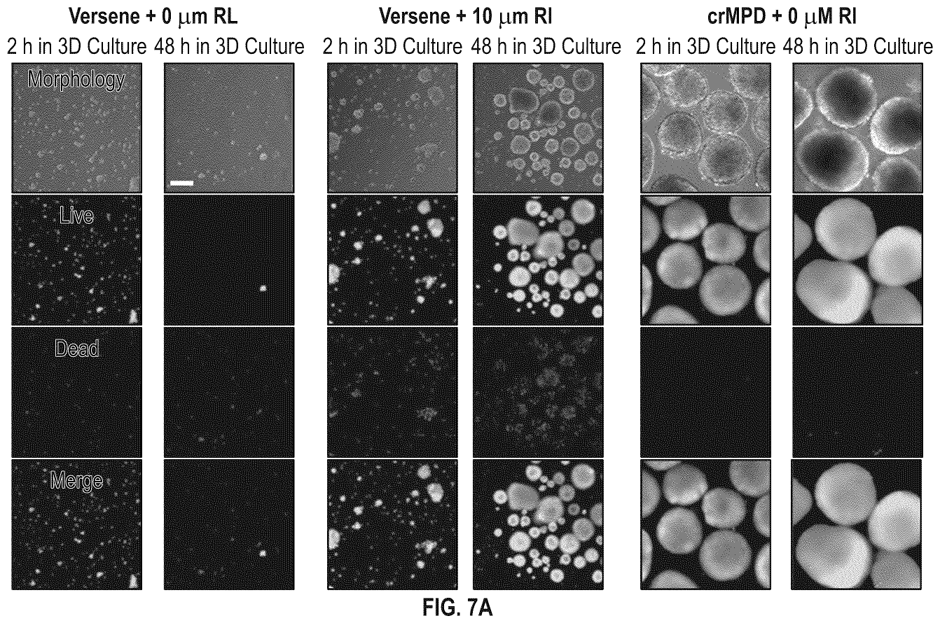

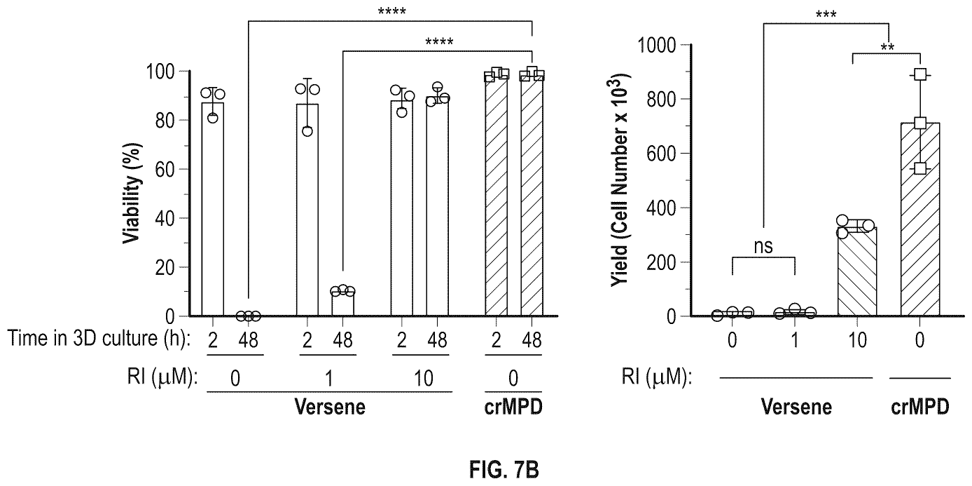

With reference now to FIGS. 7A and 7B of the DRAWINGS, the viability and yield of hiPSCs post-detachment from the crMPD and under 3D suspension culture is shown. Representative images are shown in FIG. 7A and quantitative data for the viability assessed by live/dead staining of DF19-9-11T.H hiPSCs after 2 and 48 h of 3D suspension culture post-detachment using either Versene and supplementation with 0, 1, and 10 μM RI or cold treatment of crMPD with 400 μm diameter Matrigel islands and supplementation with 0 μM RI is shown in FIG. 7B. Cell yield after 48 h of 3D suspension culture of the hiPSCs post-detachment under the aforementioned conditions is also shown in FIG. 7B, on the right. Statistical analyses were done using one-way analysis of variance (ANOVA) with Tukey's multiple comparisons test and correction. **p<0.01, ***p<0.001, ****p<0.0001, ns represents not significantly different. n=3 independent runs. Scale bar: 200 μm. Error bars represent standard deviation.

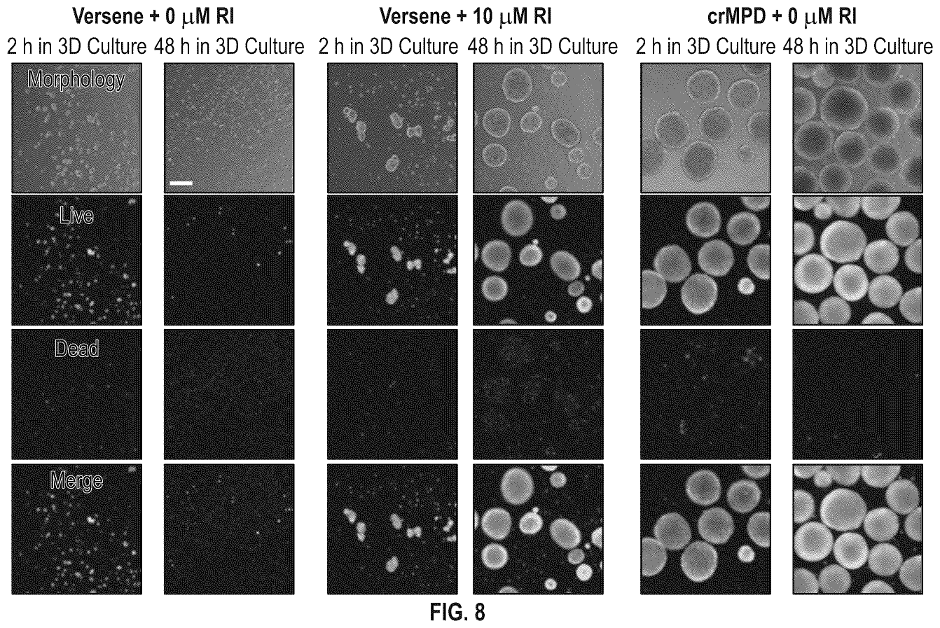

With reference now to FIG. 8 of the DRAWINGS, there is shown live and Dead Staining of IMR90-1 hiPSCs detached and 3D-cultured in three different ways. Representative images of the viability assessed by live/dead staining of hiPSCs at 2 and 48 hours post-detachment (PD) either using Versene and supplementation with 0, 10 μM RI for 3D culture or by cold treatment from crMPD with 400 μm diameter Matrigel islands and supplementation with 0 μM RI for 3D culture. Scale bar: 200 μm

As noted, representative images of the DF19-9-11T.H hiPSCs at 2 hours and 48 hours post-detachment and in 3D culture are shown in FIGS. 7A and 7B, with the quantitative viability data being shown in FIG. 7B. After 2 hours of 3D culture, the hiPSCs from all conditions show high viability (>85%). However, without RI supplementation, most of the hiPSCs detached using Versene die by 48 h in 3D culture (Versene+0 μM RI, 0.3±0.2% viability), even though most of the cells are alive after 2 hours in 3D culture (87.6±5.7%). Under this condition, although some cell aggregates could be seen in 3D culture at 2 hours, most of them dissociate into single cells by 48 h. The supplementation of RI helps cells detached with Versene to survive 3D culture, as expected. Supplementation with a small amount of RI (1 μM) prevents 10.4±0.3% hiPSCs from death at 48 hours of 3D culture, while increasing the amount of RI to 10 μM RI results in a cell viability of 90.1±3.1% at 48 h of 3D culture.

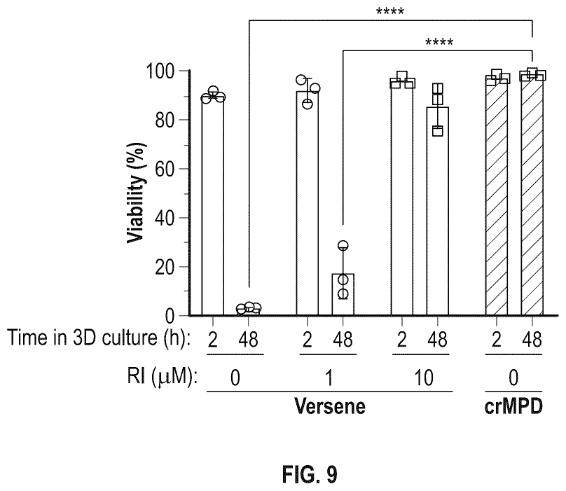

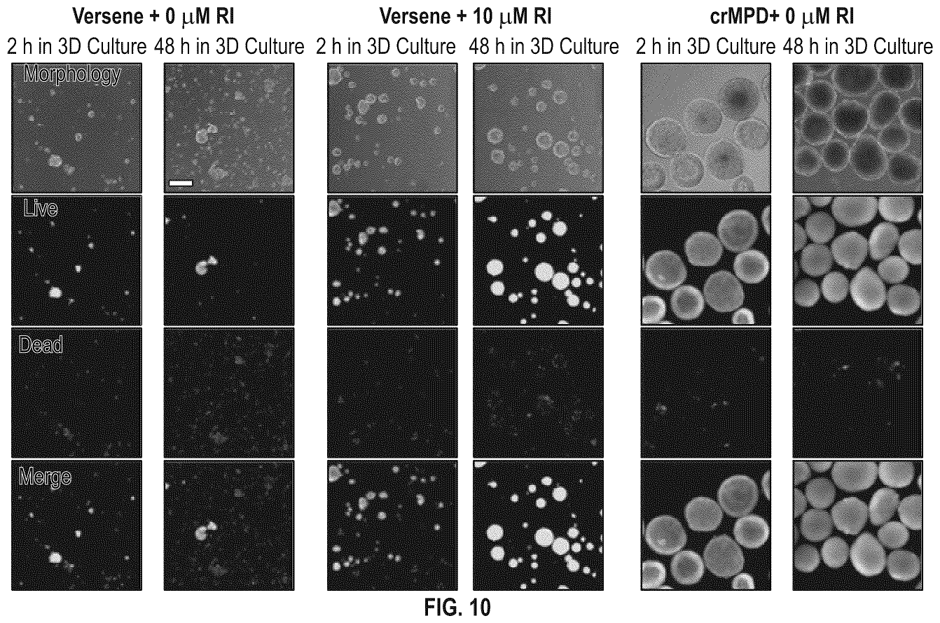

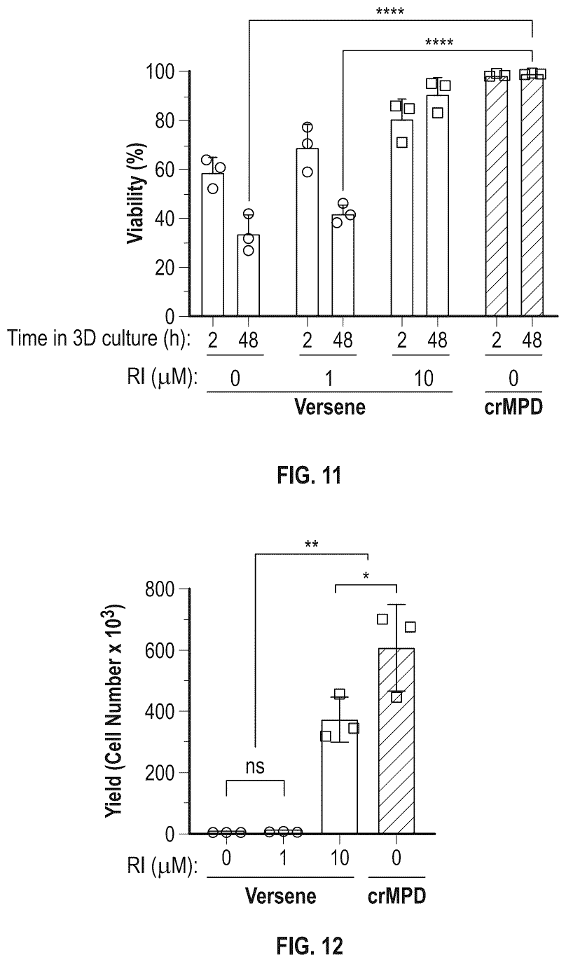

Importantly, hiPSCs detached with cold treatment from the crMPD show high viability both at 2 h (98.7±1.0%) and 48 hours (98.5±0.6%) post-detachment and 3D culture without the need of any RI. The hiPSCs detached from the crMPD show statistically significant higher viability than those from the Versene+0 μM RI and Versene+1 μM RI groups and similar (but slightly higher) viability to the Versene+10 μM RI group at 48 hours. Although both hiPSCs detached using Versene and supplemented with 10 μM RI and hiPSCs detached from the crMPD without RI are seen to mostly aggregate without many single cells after 2 hours of 3D culture that grow into larger spheroids by 48 hours, more dead single cells can be seen for the Versene-detached cells even in the presence of 10 μM RI during 3D culture. A similar trend of the cell viability at 2 hours and 48 hours is observable for two other hiPSC cell lines: IMR 90-1 cells, As shown in FIGS. 8 and 9, and the reprogrammed hiPSCs, as shown in FIGS. 10 and 11, which further confirms that crMPD can be used with cold treatment for cell detachment to improve hiPSC viability during 3D culture, independent of hiPSC types.

With particular reference to FIG. 9, there is shown quantitative data on the viability of IMR90-1 hiPSCs detached and 3D-cultured in four different ways. The viability is assessed by live/dead staining of hiPSCs at 2 and 48 h post-detachment (PD) either using Versene and supplementation with 0, 1, 10 μM RI for 3D culture or by cold treatment from crMPD with 400 μm diameter Matrigel islands and supplementation with 0 μM RI for 3D culture.

Statistical analyses were done using one-way analysis of variance (ANOVA) with Tukey's multiple comparisons test and correction. ****p<0.0001. n=3 independent runs. Error bars represent standard deviation.

With particular reference to FIG. 10 of the DRAWINGS, there are shown live and dead staining of reprogrammed hiPSCs detached and 3D-cultured in three different ways. Representative images of the viability assessed by live/dead staining of hiPSCs at 2 and 48 hours post-detachment (PD) either using Versene and supplementation with 0, 10 μM RI for 3D culture or by cold treatment from crMPD with 400 μm diameter Matrigel islands and supplementation with 0 μM RI for 3D culture. Scale bar: 200 μm.

With particular reference to FIG. 11 of the DRAWINGS, there are shown quantitative data on the viability of reprogrammed hiPSCs detached and 3D-cultured in four different ways. The viability is assessed by live/dead staining of hiPSCs at 2 and 48 hour post-detachment (PD) either using Versene and supplementation with 0, 1, 10 μM RI for 3D culture or by ice cold treatment from crMPD with 400 μm diameter Matrigel islands and supplementation with 0 μM RI for 3D culture.

Statistical analyses were done using one-way analysis of variance (ANOVA) with Tukey's multiple comparisons test and correction. ****p<0.0001. n=3 independent runs. Error bars represent standard deviation.

To determine the yield of hiPSCs from the aforementioned four different combinations of cell detachment methods and RI concentrations for 3D culture, the same number of hiPSCs were cultured in 3D suspension for 2 days, the resultant hiPSC spheroids were collected, and the spheroids were dissociated into single cells to count the cell number. The aforementioned quantitative data on the yield of DF19-9-11T.H is shown in FIG. 7B. The hiPSC yield for the condition of cell detachment using Versene without any RI supplementation for 3D culture is negligible, as few cells survive to form spheroids in 3D culture without RI, as shown in FIGS. 7A and 7B.

The addition of 1 μM RI to the Versene-detached cells for 3D culture results in a slight but not statistically significant increase of the cell yield by ~1.5 fold, compared to the condition of Versene-detachment without RI supplementation. Increasing the concentration of RI to 10 μM greatly and significantly enhances cell yield for Versene-detached cells by ~32 and ~20 folds, compared to Versene-detached cells with the 0 and 1 μM RI supplementation, respectively.

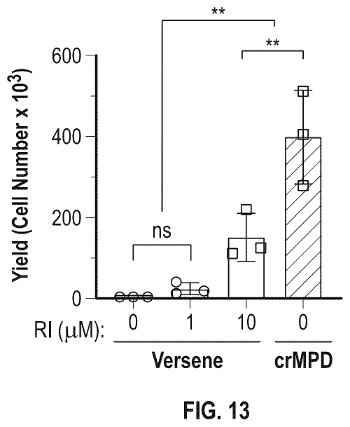

Importantly, hiPSCs detached by ice cold treatment from the crMPD without any RI supplementation for 3D culture also show a greatly and significantly enhanced (by ~2.1 fold) cell yield, compared to the hiPSCs detached using Versene and supplemented with 10 μM RI for 3D culture. With reference now to FIGS. 12 and 13 of the DRAWINGS, a similar trend in the cell yield for the four different conditions is also observable for the IMR90-1 cells and the reprogrammed hiPSCs, showing the greatly and significantly enhanced cell yield by using the crMPD with ice cold-triggered detachment of hiPSCs for 3D culture is general for all of the hiPSCs.

With particular reference now to FIG. 12 of the DRAWINGS, there are illustrated graphs of the yield of IMR90-1 hiPSCs detached and 3D-cultured in four different ways. The cell yield is assessed at 48 hour post detachment either using Versene and supplementation with 0, 1, 10 μM RI for 3D culture or by ice cold treatment from crMPD with 400 μm diameter Matrigel islands and supplementation with 0 μM RI for 3D culture.

Statistical analyses were done using one-way analysis of variance (ANOVA) with Tukey's multiple comparisons test and correction. *p<0.05, **p<0.01, and ns: not significant. n=3 independent runs. Error bars represent standard deviation.

With particular reference to FIG. 13 of the DRAWINGS, there are also illustrated graphs of the yield of reprogrammed hiPSCs detached and 3D-cultured in four different ways. The cell yield is assessed at 48 hour post detachment either using Versene and supplementation with 0, 1, 10 μM RI for 3D culture or by ice cold treatment from crMPD with 400 μm diameter Matrigel islands and supplementation with 0 μM RI for 3D culture.

Statistical analyses were done using one-way analysis of variance (ANOVA) with Tukey's multiple comparisons test and correction. **p<0.01 and ns: not significant. n=3 independent runs. Error bars represent standard deviation.

High Homogeneity in Size of hiPSC Spheroids Produced via crMPD

The size distributions of the hiPSC spheroids produced either using Versene with either 1 or 10 μM RI supplementation for 3D culture or by ice cold-triggered detachment from crMPD with no RI supplementation for 3D culture, were investigated to confirm the production of homogenous spheroids ideal for further differentiation of the cells. Both the hiPSCs detached using Versene with either 1 or 10 μM RI supplementation or from crMPD and the hiPSCs detached from crMPD with no RI supplementation were cultured in 3D suspension for 2 days.

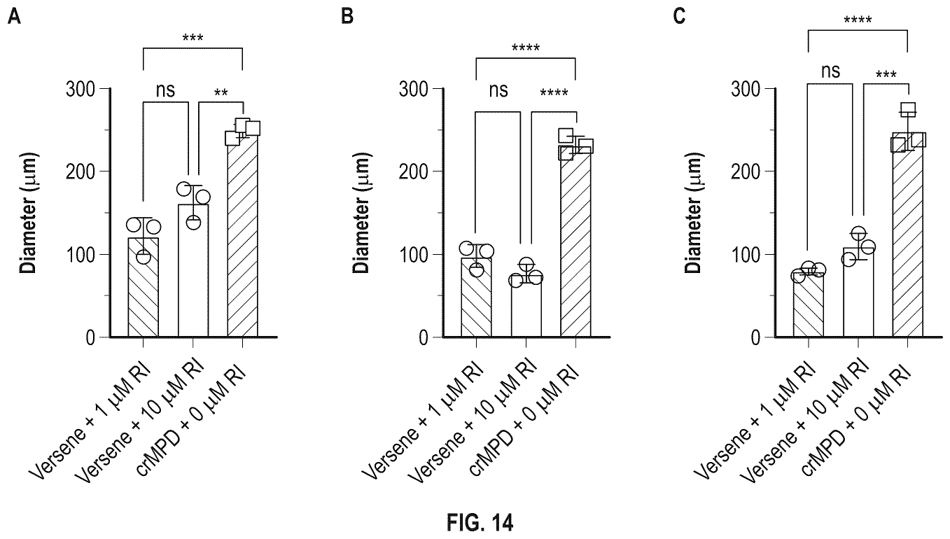

For all the three different hiPSC lines, the spheroids produced using Versene detachment with 1 and 10 μM RI supplementation for 3D culture are similar in size, and significantly smaller than the hiPSC spheroids made using the crMPD with cold-triggered detachment and without any RI for 3D culture, as illustrated in FIG. 14 of the DRAWINGS, with respective parts A, B and C set forth therein. With particular regard to FIG. 14, this illustrates the average size of the hiPSC spheroids detached and 3D-cultured in three different ways. Part A, DF19-9-11T.H. hiPSCs; Part B, IMR90-1 hiPSCs; and Part C, Reprogrammed hiPSCs. Statistical analyses were done using one-way analysis of variance (ANOVA) with Tukey's multiple comparisons test and correction. **p<0.01, ***p<0.001, ****p<0.0001, and ns: not significant. n=3 independent runs. Error bars represent standard deviation.

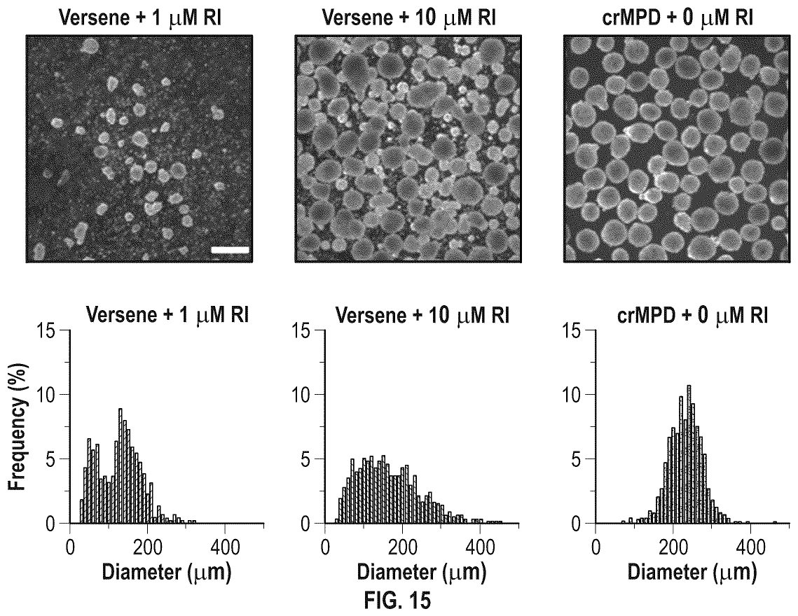

Furthermore, the distribution of the spheroid diameter from the three conditions differs greatly. Representative images of the DF19-9-11T.H hiPSC spheroids produced from Versene-detached hiPSCs supplemented with 1 or 10 μM IR for 3D culture and cold-detached hiPSCs from crMPD without RI supplementation for 3D culture, are shown in FIG. 15, particularly the upper images therein. The hiPSC spheroids formed by using Versene detachment vary greatly in size due to uncontrolled colony size, as evidenced by the representative images and the accompanying wide size distributions shown in the histograms in FIG. 15 below the respective illustrations. Importantly, the hiPSC spheroids generated with the crMPD are round and very homogeneous in size with a narrow gaussian distribution as seen in FIG. 15.

With particular reference to FIG. 15, the aforesaid homogeneous size of hiPSC spheroids produced by using the crMPD are set forth in three ways, with representative images, and associated histograms showing size distribution, of DF19-9-11T.H hiPSC spheroids produced after detachment using either Versene supplemented with RI (1 or 10 μM) or cold treatment of the crMPD supplemented with 0 μM RI. n=3 independent runs. Scale bar: 500 μm.

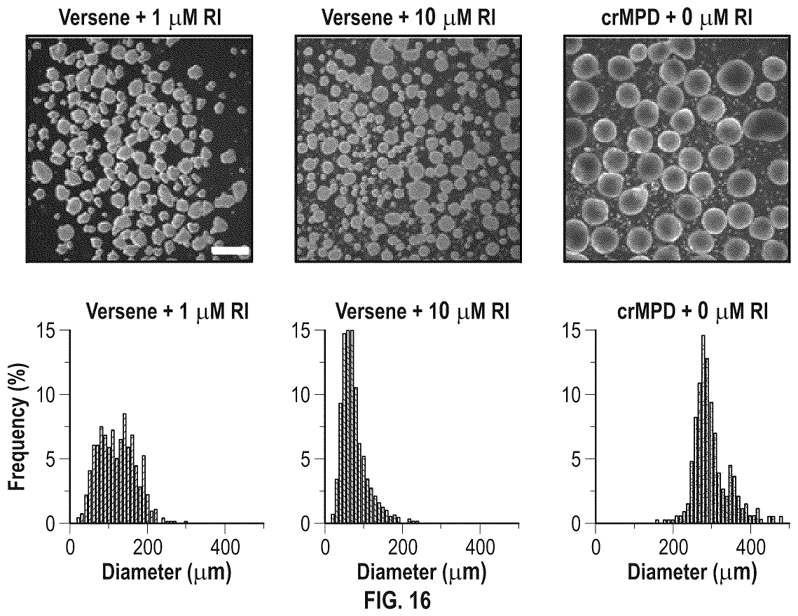

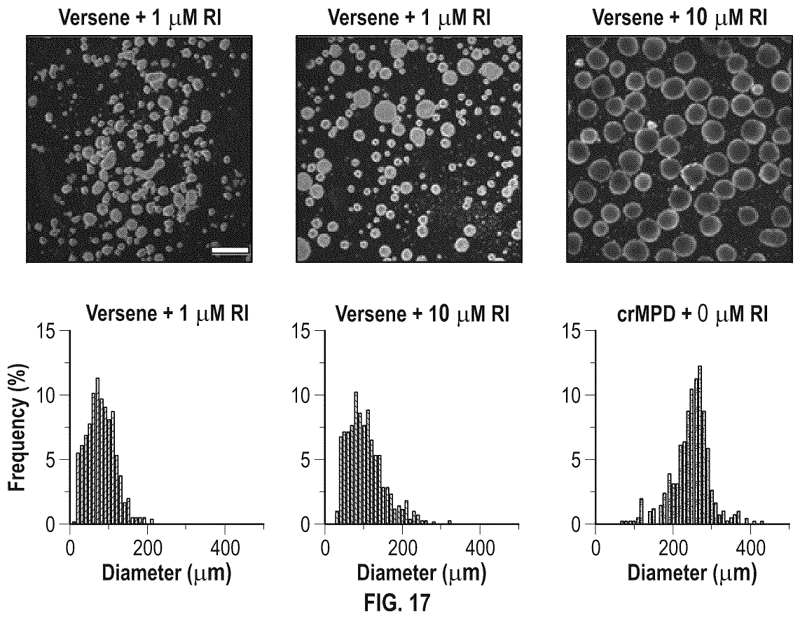

With reference now to FIGS. 16 and 17 of the DRAWINGS, a similar trend in the hiPSC spheroid homogeneity is also observable for the IMR90-1 cells and the reprogrammed hiPSCs, showing the improved homogeneity of the resultant 3D spheroids by using the crMPD with ice cold-triggered detachment of hiPSCs for 3D culture is general for all of such hiPSCs so generated.

With particular reference to FIG. 16 of the DRAWINGS, the aforementioned homogeneous size of IMR90-1 hiPSC spheroids are again produced in three different ways, with representative images, and associated histograms showing size distribution, of hiPSC spheroids produced after detachment either using either Versene supplemented with 1 or 10 μM RI for 3D culture (two conventional ways) or by ice cold treatment of the crMPD supplemented with 0 μM RI for 3D culture (crMPD-based approach). The IMR90-1 hiPSC spheroids produced by the cdMPD-based approach are more homogeneous and larger than that produced by the two conventional ways. n=3 independent runs. Scale bar: 500 μm.

With particular reference to FIG. 17 of the DRAWINGS, the aforementioned homogeneous size of reprogrammed hiPSC spheroids are also produced in three different ways, with representative images, and associated histograms showing size distribution, of hiPSC spheroids produced after detachment either using either Versene supplemented with 1 or 10 μM RI for 3D culture (two conventional ways) or by ice cold treatment of the crMPD supplemented with 0 μM RI for 3D culture (crMPD-based approach). The reprogrammed hiPSC spheroids produced by the crMPD-based approach are more homogeneous and larger than that produced by the two conventional ways. n=3 independent runs. Scale bar: 500 μm.

High Pluripotency of hiPSC Spheroids Produced via crMPD with Cold-Triggered Cell Detachment

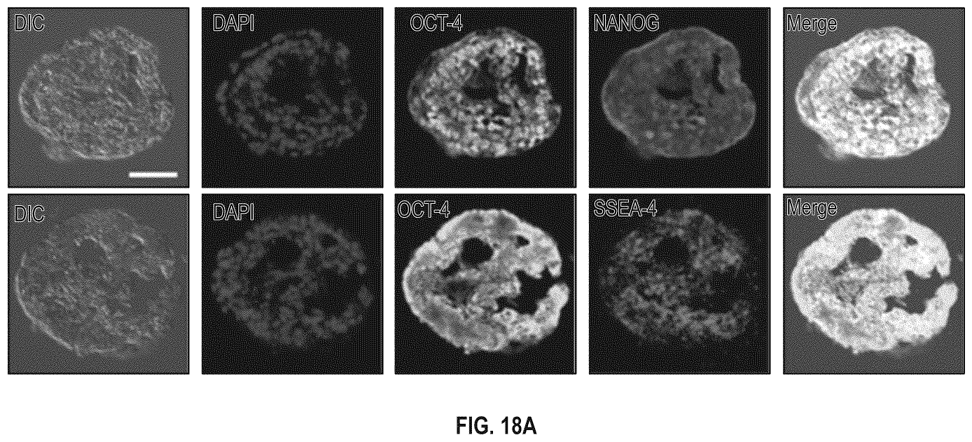

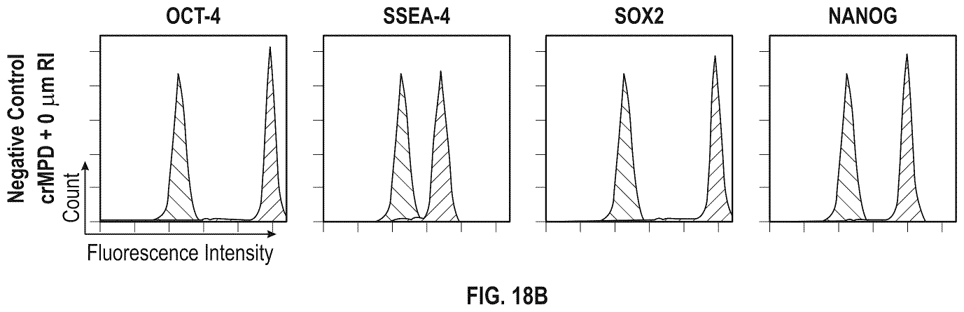

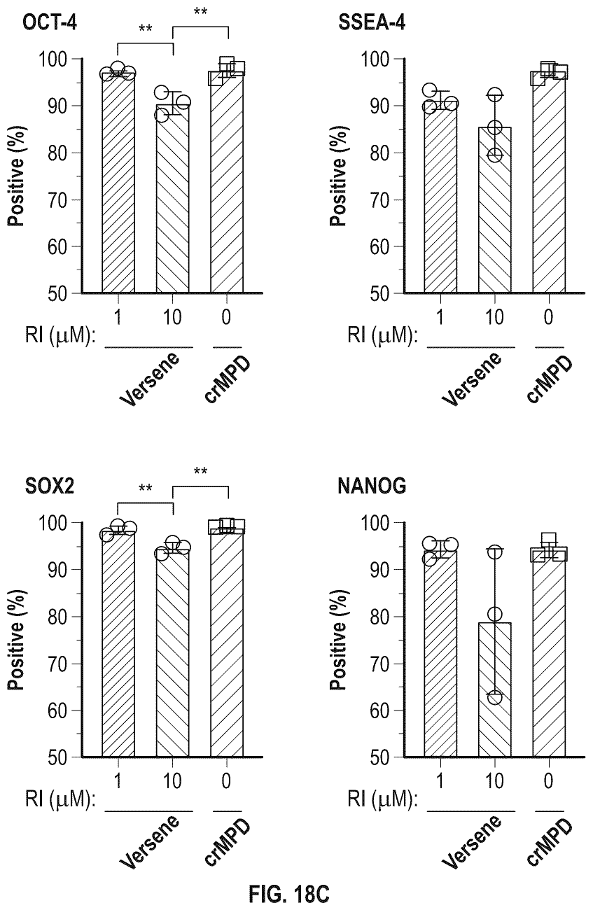

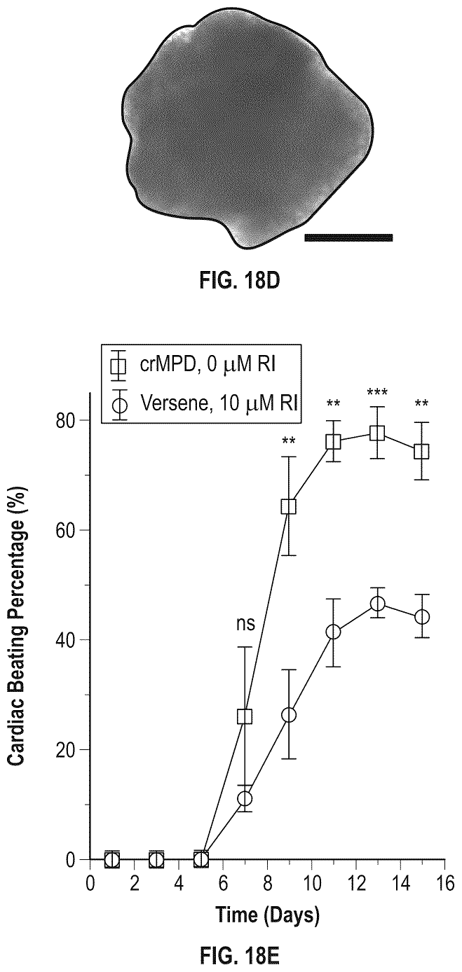

To evaluate the quality of hiPSC spheroids produced using the crMPD, the expression of four pluripotency markers (OCT-4, SSEA-4, NANOG, SOX2) was investigated. Immunostaining shows that DF19-9-11T.H hiPSC spheroids produced using the crMPD-based method are highly positive for OCT-4, SSEA-4, NANOG, and SOX2, as illustrated in FIGS. 18A-18E of the Drawings. Flow cytometry analyses were used to quantitatively evaluate the expression of the four pluripotency markers of hiPSC spheroids produced using the crMPD without RI supplementation compared to hiPSC spheroids produced using Versene detachment and supplementation with 1 or 10 μM RI, as shown in FIG. 18A.

With particular reference to FIGS. 18A-18E of the DRAWINGS, the high pluripotency of hiPSCs produced by using the crMPD are illustrated. In FIG. 18A, there are shown representative images of DF19-9-11T.H hiPSC spheroids produced by using the crMPD, showing their high expression of pluripotency markers OCT-4, NANOG, SOX2, and SSEA-4 via immunostaining. DIC: differential interference contrast. Scale bar: 50 μm.

In FIG. 18B, there are shown representative flow cytometry peaks of the pluripotency markers OCT-4, SSEA4, SOX2, and NANOG of cells from hiPSC spheroids produced by cold detachment from crMPD and supplementation with 0 μM RI for 3D culture of the DF19-9-11T.H hiPSCs. The negative controls (secondary antibody only, no primary antibody) are shown in light blue.

In FIG. 18C, there are shown quantitative data from flow cytometry analysis showing expression of pluripotency markers OCT-4, SSEA4, SOX2, and NANOG of cells in hiPSC spheroids produced either by Versene detachment supplemented with 1 or 10 μM RI for 3D culture or by cold detachment from crMPD and supplementation with 0 μM RI for 3D culture.

In FIG. 18D, there is shown a representative image of cardiac organoids (on day 15 of differentiation) differentiated from hiPSC spheroids produced by the crMPD-based method. Scale bar: 250 μm.

In FIG. 18E, there is shown a graph of a beating percentage of cardiac organoids produced from hiPSC spheroids grown from hiPSCs obtained via either Versene detachment with 10 μM RI for 3D culture or cold detachment from crMPD with 0 μM RI for 3D culture. Statistical analyses were done using one-way analysis of variance (ANOVA) with Tukey's multiple comparisons test and correction for panel C and two tailed unpaired t-test for panel E. *p<0.05, **p<0.01, ***p<0.001, and ns: not significant. n=3 independent runs. Error bars represent standard deviation.

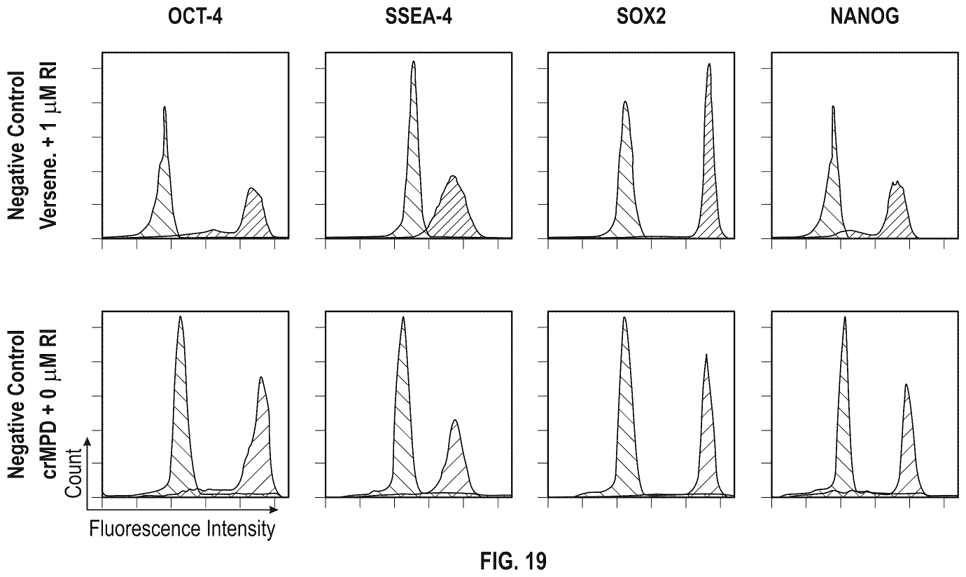

With reference now to FIG. 19 of the DRAWINGS, there are shown representative flow cytometry peaks showing the expression the pluripotency markers in hiPSC spheroids produced by Versene detachment. The DF19-9-11T.H hiPSC spheroids detached by Versene supplemented with 1 or 10 μM RI for 3D culture are shown in the top and bottom rows, respectively. OCT-4, SSEA-4, SOX2, and NANOG are pluripotency markers. The negative controls (secondary antibody only, no primary antibody) are shown in light green (1 μM, top row), dark green (10 μM, bottom row).

Representative peaks of pluripotency markers from all three conditions are given in FIG. 5B and also shown in FIG. 19. Although little overlap can be seen between the isotype control and the experimental fluorescence intensity peaks of all the four pluripotency markers for all the three different methods, the experimental fluorescence intensity peaks for the crMPD group are narrower and with clearer separation from the corresponding isotype peaks, as shown in FIG. 18B, than the fluorescence intensity peaks for the Versene detachment groups with either 1 or 10 μM RI supplementation, as shown in FIG. 19, suggesting better quality of the hiPSCs obtained with the crMPD-based approach than the two conventional methods. This is further confirmed by the quantitative data shown in FIG. 18C for the percentage of hiPSCs that are positive for the four markers.

The hiPSCs from the crMPD group are >95% positive for all the four pluripotency markers with little variation. The expression of all the four pluripotency markers is higher in hiPSCs from the crMPD group than the Versene detachment method with 10 μM RI, and the difference is significant for OCT-4 and SOX2. The same trend is observable when comparing the hiPSC spheroids produced by Versene detachment with 1 μM RI to 10 μM RI. Although there is no significant difference between the expression of pluripotency markers for the 1 μM RI Versene-detached hiPSC spheroids and the 0 μM RI crMPD-produced spheroids, the viability, yield, and homogeneity of the hiPSCs from the crMPD group is much better than the hiPSCs obtained by Versene detachment with 1 μM RI for 3D culture shown in FIGS. 7 and 15 discussed hereinabove.

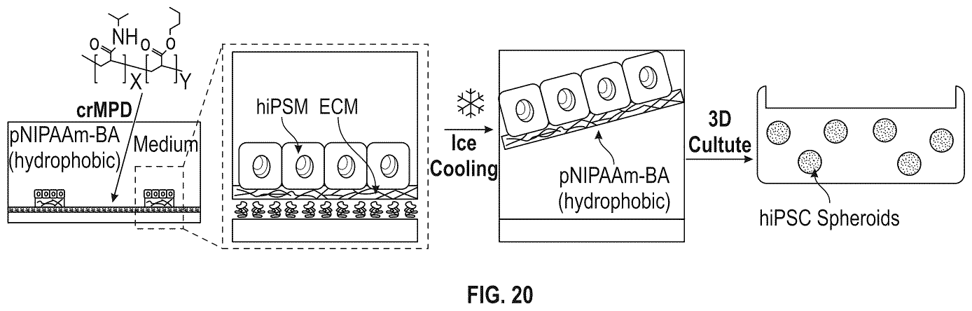

With reference now to FIG. 20 of the DRAWINGS, there is illustrated a general paradigm of the present invention where cold-triggered chemical-free cell detachment enables convenient ROCK inhibitor-free mass production of high-quality and homogeneous 3D human iPSC spheroids. As discussed, the cold-responsive micropatterned dish (crMPD) is fabricated by spin-coating a cell culture dish with a cold-responsive polymer and then microcontact printing cell attachment micropatterns on that surface. Later, the micropatterns, with the stem cells grown thereon, can be physically detached as a whole by simply ice cooling without using any chemical agents, enabling convenient large-scale production of homogeneous 3D hiPSC spheroids with high viability, yield, and quality, as described in detail herein.

Improved Cardiac Differentiation Capacity of hiPSC Spheroids Produced via crMPD

To further validate the significantly improved quality of the hiPSC spheroids produced by crMPD without RI supplementation for 3D culture, compared to Versene detachment and supplementation with 10 μM RI for 3D culture, differentiation of the hiPSC spheroids into beating cardiac organoids was conducted by using a commercially available PSC cardiomyocyte differentiation kit.

Cardiomyocyte differentiation was chosen because the quality of differentiation could be visually assessed and conveniently quantified, as functional cardiac organoids exhibit strong beating when viewed under a microscope. Although strong beating is observable for cardiac organoids produced from both groups after 15 days of differentiation, as shown in FIG. 18D, the crMPD group has a significantly higher percentage of beating organoids starting from day 9 of differentiation than the group of Versene detachment and 10 μM RI supplementation, as shown in FIG. 18E. These data confirm that hiPSC spheroids produced via crMPD without RI supplementation for 3D culture have greater differentiation potential than the hiPSC spheroids grown from the conventional way using Versene for detachment and supplementation of 10 μM RI for 3D culture.

### Discussion

In this work, the crMPD, as discussed hereinabove in connection with FIGS. 4. 5A and 5B, and 6, a device for convenient and mass production of high quality hiPSC spheroids with narrow size distribution (FIGS. 4, 5C and 5DD, and FIGS. 16 and 17), is developed using a facile microcontact printing method (FIG. 1A). The specific pNIPAAm-BA copolymer chosen for making the cold-responsive surface has an LCST of about 0-12 °C, about 2-8 °C or about 4-8 °C, which means that cells can only be released from the device upon cooling below that temperature. The LCST is much lower than room and cell-incubation temperatures (~22 °C and 37 °C), so that cells are not accidentally released during routine media changes and viewing under microscope at room temperature. In this study, the crMPD is cooled on ice to release the hiPSC colonies (FIGS. 1B, 5B and 5C), which is an easily acceptable cooling method that does not require any specialized reagents or equipment except a fridge or ice maker that is usually available in biomedical research labs and clinical facilities. Additionally, the release of cells at this ice-cold temperature may help to slow cell metabolism and oxygen demand, which could have a beneficial effect on the cells and lessen the possible adverse impact of conventional detachment methods that usually involve incubation at 37 °C for a few minutes.

A PDMS-stamp-based microcontact printing approach was used here to pattern the ECM on the cold-responsive surface for cell attachment. The cell attachment matrix used here is Matrigel, but other proteins, such as collagen, vitronectin, and lamnin, could be used for more defined cell culture conditions. The microcontact printing approach is easily modifiable, and the design of the stamp can be altered to create different size, shape, and patterns of ECM for transferring onto the cold-responsive surface. The PDMS stamp can also be easily reused many times after simple cleaning and sterilization in 70% aqueous ethanol solution.

As discussed and illustrated, the design in this study used circular Matrigel islands of 100 μm and 400 μm in diameter for cell attachment, but the diameter of these islands could be modified by creating a new PDMS stamp design. The micropattern of the crMPD can be tuned to produce hiPSC colonies of different sizes for different differentiation applications. For example, one study found that hiPSC spheroids 500-600 μm in diameter are optimal for hepatic differentiation. The crMPD can therefore be customized to produce hiPSC spheroids of the desired size for the desired differentiation protocol. Because the crMPD could be designed with specific hiPSC colony sizes (400 μm in this case), the hiPSCs only needed to be cultured in 3D suspension for 2 days after release from the crMPD, to self-assemble and grow into the desired size. This results in spheroids with a narrow size distribution, as shown hereinabove in connection with FIGS. 15-17. However, the hiPSC colonies detached via Versene did not have such a method to control the initial size, and the initial colonies produced by this method varied greatly in size, much more so than the hiPSC colonies detached from the crMPD. Because the size of the initial colonies is difficult to control with the Versene detachment, the final hiPSC spheroids varied greatly in size, with a very wide size distribution (FIGS. 15-17), which is not ideal for differentiation applications. The crMPD can be customized to design patterns for producing hiPSC spheroids of the desired size, shortening the time needed to self-assemble in suspension culture and presumably reduce uncontrolled fusion/merging of cell aggregates during prolonged suspension culture.

The hiPSC spheroids produced via the crMPD show high viability and yield (FIG. 7), a narrow size distribution (FIGS. 15-17), and high expression of pluripotency markers (FIG. 8). The hiPSC spheroids produced via the crMPD were compared to hiPSC spheroids produced via a conventional method for generating hiPSC spheroids: detachment of 2D hiPSCs using Versene and subsequent suspension culture of the detached hiPSC colonies until they assemble into hiPSC spheroids, helped by the addition of RI to the medium.

RI works to prevent anoikis-induced apoptosis that may occur during detachment/dissociation into single hiPSCs associated with Versene-based detachment. Increasing the amount of RI in the media during 3D suspension culture of the Versene-detached hiPSCs indeed helps the cells to survive, as evidenced by the higher viability and yield of the 10 μM RI group compared to the 1 μM RI group (FIGS. 7-13). However, the higher concentration of RI shows unwanted effects on hiPSC pluripotency, as the 10 μM RI group shows a trend of significantly lower expression of two pluripotency markers compared to the 1 μM RI group (FIG. 18C), which has also been shown before[9a].

Because the hiPSC colonies released from the crMPD are not dissociated into single cells and presumably should maintain ECM and cell-cell connections in each colony from 2D culture, they are able to form into high quality hiPSC spheroids without the need for RI with high viability and significantly higher yield (FIGS. 3C, S5-10) and cardiac differentiation capacity (FIG. 5D) than the 10 μM RI Versene-detached hiPSC spheroids and at least comparable quality to the 1 μM RI Versene-detached hiPSC spheroids in terms of pluripotency marker expression (FIG. 5C). In other words, the crMPD method combines all the advantages of the Versene-detachment methods with 1 and 10 μM RI while eliminating their drawbacks, which can be quite deleterious.

### Some Conclusions

A cold-responsive, micropatterned device (crMPD) is developed for cell culture to generate high quality and homogeneous hiPSC spheroids. The hiPSCs can successfully attach and grow on the micropatterned cell attachment design that is microprinted onto the cold-responsive pNIPAAm-BA coated surface. The hiPSC growth is confined only to areas of micropatterned Matrigel, so that defined hiPSC colonies of a specific size are generated. These hiPSC colonies can be easily released from the crMPD surface by cooling the device on ice (about 0 °C), which is below the LCST of the pNIPAAm-BA (4-8 °C). Under 3D suspension culture, the released hiPSC colonies self-assemble into 3D hiPSC spheroids with high viability, yield, and pluripotency without the need for any RI supplementation in the culture medium. Because the hiPSC colonies are patterned to be the same size on the surface of the crMPD, the resulting hiPSC spheroids have a narrow size distribution, ideal for downstream differentiation of the hiPSC spheroids into the desired cell lineages. This method can be used for convenient mass production of homogeneous, high-quality hiPSC spheroids for lineage-specific differentiation, which is invaluable for facilitating the advance of hiPSC-based research and personalized medicine.

### Exemplary Methods

Synthesis of pNIPAAm-BA

The pNIPAAm-BA with an LCST range of about 4-8 °C was synthesized by controlling the polymerization of NIPAAm (ThermoFisher Scientific, Waltham, MA, USA) and BA (Sigma-Aldrich, Rockville, MD, USA). Briefly, the molar ratio of NIPAAm to BA was set at 83:17, where 16.6 mmol NIPAAm and 3.4 mmol BA were added to the reaction flask in 10 mL of dioxane (Sigma-Aldrich). After purging with nitrogen gas for 30 min, 0.5% (w/v) 2,2-Azobis(2-methylpropionitrile) (AIBN, Sigma-Aldrich) in 5 mL of dioxane was added dropwise into the reaction flask. The reaction flask was then heated to about 70 °C and stirred at about 400 rpm under nitrogen for about 12 h.

The polymerization was stopped by exposing the flask to air and cooling down to room temperature (~22 °C). Afterwards, the resultant sample was precipitated in excess ice-cold diethyl ether (Fisher Scientific, Waltham, MA, USA) and filtered through a filter paper (Diameter: 12.5 cm, VWR, Radnor, PA, USA). After dissolving in tetrahydrofuran (Fisher Scientific) (~10 mL) and precipitating in ice-cold diethyl ether for three times, the synthesized polymer was dried overnight under vacuum. The synthesized pNIPAAm-BA has a number average molecular weight (Mn) of 78,000 Da, polydispersity index (PDI) of 1.6, and molar ratio (x:y, x for NIPAAm and y for BA) of 5.8±0.1.

Coating of pNIPAAm-BA on the Cell Culture Surface in Petri Dish

To create a pNIPAAm-BA coating on the petri dish, a 5% (w/v) solution of pNIPAAm-BA in ethanol was prepared. A total of 200 μL (by default) of the solution was dispensed onto a 35 mm Nunc™ Cell Culture/Petri Dishes (product #: 153066, ThermoFisher Scientific) for spin coating. The two-step procedure for spin-coating the dish was: first, spinning for 15 s at 150 RPM with an acceleration of 100 RPM/s and secondly, spinning for 30 s at 2000 RPM with an acceleration of 300 RPM/s. The pNIPAAm-BA coated dishes were fully dried in a biosafety cabinet and sterilized by ultraviolet (UV) light for 20 min before use.

### SEM and EDXS

For SEM imaging, dishes partially coated with pNIPAAm-BA were cut into small pieces of 1 cm² and attached on the SEM sample mount. The samples were coated with 10 nm thick Au/Pd using a sputter coater (CCU-010 HV, Safematic, Zizers, Switzerland). Afterwards, the SEM imaging and EDXS mapping and line scanning of the samples were conducted with a combined SEM and EDXS system (SU-70 FEG, Hitachi, Tokyo, Japan) at 10 kV.

### Contact Angle Measurement

For measuring contact angle, a goniometer (L2004A1, Ossila BV, Leiden, Netherlands) was used to take images of water droplets (10 μL) on uncoated cell culture dishes and the pNIPAAm-BA coated dishes. The images were analyzed using ImageJ (v1.52q, NIH, USA).

### Making the PDMS Stamp for Micropatterning of ECM

The design of micropatterning stamps (both 100 μM and 400 μM in diameter) was created using AutoCAD (Autodesk, Mill Valley, CA, USA) and a mask of the design was printed by CAD/Art Services Inc. (Brandon, OR, USA). To create the mold, SU-8 2050 photoresist (Kayaku Advanced Materials Inc., Westborough, MA, USA) was spin-coated onto a silicon wafer (University Wafer, South Boston, MA) and soft baked on hot plates at 65 and 95 °C for 5 and 20 min, respectively. The wafer was then exposed to UV light through the patterned mask using a MA-4 Mask Aligner (Karl Suss, Munich, Germany). Next, the wafer was hard baked at 65 and 95 °C for about 5 and 10 min, respectively, and developed using SU-8 developer (Kayaku Advanced Materials, Inc.) to expose the patterned features of crosslinked SU-8. The spin speeds, baking parameters, and exposure energy were all determined based on the SU-8 2000 series datasheet from the manufacturer. The stamp was then made via soft lithography using this mold. The SYLGARD 184 Silicone Encapsulant Clear polydimethylsiloxane (PDMS, Dow, Midland, MI, USA) prepolymer and its curing agent (Dow) were mixed at a 10:1 (w/w, prepolymer:curing agent) and poured onto the mold. The PDMS on the mold was degassed in a vacuum and baked in a 75 °C oven for about 2 hours to cure/crosslink the polymer. The PDMS stamp 101 was carefully cut and peeled out of the mold and subsequently sanitized in 70% ethanol for 30 minutes by sonication.

Micropatterning Cold-Responsive Surface with PDMS Stamp

PDMS stamps 101 were first thoroughly dried with nitrogen gas. The PDMS stamps were then plasma-treated using a PDC-32G plasma cleaner (Harrick Plasma, Ithaca, NY, USA) for 3 min in sterile conditions. PDMS stamps were inked with Matrigel (Corning, Corning, NY, USA) diluted in DMEM-F12 (Gibco, Gaithersburg, MD, USA) (20 μg/mL) and kept in biosafety cabinet on ice. The inking process for the PDMS stamp can be seen in FIG. 1A. After 15 min of inking, excess solution was aspirated, and the stamp was washed with deionized water to remove excess Matrigel not adsorbed onto the PDMS stamp surface. Nitrogen gas was blown onto the PDMS stamp to remove the excess liquid before used for printing. Meanwhile, the pNIPAAm-BA coated dish 105 was plasma-treated for 5 min in sterile conditions. Then, the inked PDMS stamp 101 was placed feature-side down on the pNIPAAm-BA coated dish and a 50 g weight with the same area of the PDMS stamp was placed on top to ensure full contact between the inked features on the stamp and the pNIPAAm-BA coated dish. After 30 minutes of incubation at about 37 °C and about 5% CO₂, the dish was ready for seeding cells. To better visualize the ECM pattern on the pNIPAAm-BA coated surface, a mixture of Rhodamine B (Sigma-Aldrich, 1 μg/mL) and Matrigel (20 μg/mL) was used to ink the PDMS stamps, and the same procedure was carried out to pattern the Rhodamine B/Matrigel mixture on the pNIPAAm-BA coated surface. Images of the micropatterned surface were taken using a fluorescence microscope to visualize the pattern.

### Cell Culture

This study used three different human iPSC cell lines. DF19-9-11T. H (reprogrammed by a nonviral approach) and IMR90-1 (reprogrammed by lentivirus infection) were purchased from WiCell (Madison, WI, USA). An additional iPSC cell line was derived from human primary dermal fibroblast (HPF, PCS-201-102) from ATCC (Manassas, VA, USA) using the nonviral ReproRNA™-OKSGM Kit (STEMCELL Technologies, Vancouver, BC, Canada) by following the manufacturer's instructions.

Briefly, HPF below passage five was cultured in medium containing DMEM (Gibco) supplemented with 10% fetal bovine serum (FBS, Sigma-Aldrich), 1 mM 1-glutamine (Invitrogen, Carlsbad, CA), and 1% MEM non-essential amino acids (NEAA, Thermofisher Scientific). HPF were then trypsinized and seeded overnight on a Matrigel coated 6-well plate at 100,000 cells per well. HPF were then switched to growth medium containing Advanced DMEM (Gibco), supplemented with 10% FBS, 1 mM 1-glutamine, and recombinant B 18R protein (STEMCELL Technologies) at 175 ng/ml; and incubated for 30 minutes.

Afterward, HPF were transfected with a cocktail of RNA encoding iPSC transcription factors (ReproRNA, STEMCELL Technologies) according to the manufacturer's protocol. Transfected HPF were left in the growth medium containing the transfection cocktail overnight and then selected for by culturing the cells in growth medium containing 0.8 μg/mL puromycin (STEMCELL Technologies) for 5 days. On day 6, cells were switched to growth medium without puromyocin for two days. On day 8, cells were switched to ReproTESR (STEMCELL Technologies) medium containing 175 ng/ml of B 18R protein for 7 days. On day 15, cells were switched to ReproTESR containing no B 18R protein. On day 28, the cells were switched to StemFlex medium (Gibco) until visible colonies were formed. Colonies were then picked using an 20-gauge needle and seeded on a Matrigel (Corning) coated 6-well dish and cultured in StemFlex containing 10 μM RI (Selleck Chemicals, Houston, TX, USA) and incubated overnight.

For 2D culture, all three hiPSC cell lines were maintained in StemFlex medium on Matrigel-coated 6-well plates (1×106 cells in 1.5 mL of medium per well) at about 37 °C and 5% CO₂ with daily medium change. The cells were passaged twice a week at a ratio of 1:4 or 1:5 with Versene (ThermoFisher Scientific) detachment.

To obtain traditional 3D hiPSC spheroids (using Versene and RI), hiPSC colonies under 2D culture at 80% confluency were detached using Versene. The detached hiPSCs were filtered through a 400 μm cell strainer (Millipore Sigma, Rockville, MD, USA) as control of the 400 μm micropattern, and re-suspended in StemFlex with 0, 1 or 10 μM RI and 0.35% (v/v) methylcellulose (R&D Systems, Minneapolis, MN, USA). The cells were transferred into a 1 0cm polystyrene petri dish for 2 days of suspension culture with RI. To generate hiPSC spheroids using the crMPD without RI, hiPSC colonies under 2D culture at 80% confluency were detached and dissociated using Versene.

The detached cells were then plated on the crMPD at 1×104 cells/cm2 and allowed to attach overnight. Cells were cultured on the crMPD for 4 days with StemFlex medium, and the medium was changed every day. Confluent hiPSCs on the crMPD were then detached by cooling the dish on ice (~0 °C) for ~15 minutes. The detached hiPSCs were then collected via pipetting, transferred to a 5 mL centrifuge tube, allowed to sink to the bottom of the tube (~5 min), and washed with pre-cooled (on ice) StemFlex to remove any dissolved pNIPAAm-BA in the solution. Cells were transferred to StemFlex supplemented with 0.35% methylcellulose and cultured in a polystyrene petri dish for 2 days in 3D suspension culture to allow for spheroid formation.

Cryomicroscopy for Cell and Matrigel Detachment on crMPD

The pNIPAAm-BA was spin coated onto a quartz sample holder and the ECM pattern was micropatterned onto it as described above. To visualize cell detachment, hiPSCs were seeded and allowed to attach overnight onto micropatterned Matrigel islands. After 2 days of growth for 100 μm crMPD and 4 days of growth for 400 μm crMPD, the quartz window was placed on the cryostage (Linkam Scientific, Surrey, UK) to allow for controlled cooling of the sample while imaging the cells. Pre-cooled (in about 4 °C) StemFlex medium was added to the stage. The sample was cooled from about 10 °C to 0 °C at a rate of about 1 °C/min, then held at about 0 °C for 10 minutes. Images were taken every other second during the holding time at about 0 °C to visualize hiPSCs and ECM patterns detaching from the substrate.

Viability and Yield of Cells Detached Using Versene Versus crMPD Method

The viability of cells detached using Versene with 0, 1, or 10 μM RI or via cold treatment on the 400 μm Matrigel island in crMPD with 0 μM RI was evaluated by live/dead staining of the detached cells. After either 2 hours or 48 hours of 3D suspension culture post-detachment, the cells were stained with calcein AM (1 μM) and propidium iodide (PI, 1 μg mL−1) to visualize live (green stain) and dead (red stain) cells, respectively. Cells were incubated with the dye for 10 min at 37 °C and 5 % CO₂ before imaging with a Zeiss (Oberkochen, Germany) LSM710 microscope to capture green and red fluorescence. Cell viability is calculated as the percent of the area of live cells/cell aggregate area out of the total area of cells/cell aggregates.

To determine the cell yield based on detachment method, the same number of hiPSCs were detached from 2D culture using Versene and 1 or 10 μM RI or using cold treatment on the 400 μm Matrigel island in crMPD and cultured in 3D suspension for 2 days. Afterwards, the cells that formed aggregates were collected by centrifugation at 300 RPM for 3 min. Cells were dissociated into single cells using 0.25% trypsin-EDTA (ThermoFisher Scientific) and counted using a hemacytometer.

Flow Cytometry for Evaluating hiPSC Pluripotency Markers

For flow cytometry, the DF19-9-11T.H hiPSC spheroids made by either Versene detachment with 1 or 10 μM RI supplementation or cold treatment on the 400 μm Matrigel island in crMPD with 0 μM RI supplementation, were collected after two days of 3D suspension culture to create hiPSC spheroids. The spheroids were dissociated to single cells using 0.25% trypsin-EDTA for 5 minutes at 37 °C. The cells were fixed with 4% paraformaldehyde (PFA, ThermoFisher Scientific) in phosphate buffered saline (PBS, Thermofisher Scientific) for 10 min at RT, permeabilized with 0.1 % Triton-X (Sigma-Aldrich) for 5 min, blocked for non-specific binding using 3% bovine serum albumin (BSA, Sigma-Aldrich) in 1×PBS for 1 h at RT, and incubated with primary antibodies (1:500 dilution using 3% BSA solution) overnight at 4 °C.

The next day, cells were incubated with secondary antibody (1:1000 dilution using 3% BSA) for 1 hour at RT in the dark. The cells were washed with 1×PBS and analyzed using a BD Biosciences (San Jose, CA, USA) FACSCelesta Flow cytometer. The primary antibodies were NANOG (CST #4893, Cell Signaling Technologies, Danvers, MA, USA), OCT4 (ab19857, Abcam, Cambridge, UK), SOX2 (ab93689, Abcam), and SSEA-4 (CST #4755, Cell Signaling Technologies). The secondary antibodies were either goat anti-mouse (A-28175, ThermoFisher Scientific) or goat anti-rabbit Alexa Fluor™ 488 tagged antibodies (A-11008, ThermoFisher Scientific). All flow data were analyzed using Flowjo v10 (BD Bioscience).

Cryosectioning and immunostaining for evaluating hiPSC pluripotency markers The DF19-9-11T.H hiPSC spheroids were fixed in 4% PFA in 1×PBS at 4 °C overnight. The fixed spheroids were transferred to 10% sucrose (Sigma-Aldrich) in PBS for 4 hours and then 15% sucrose in PBS for 4 hours. Afterwards, the spheroids were embedded in Optimal Cutting Temperature (OCT, Tissue-Tek Sakura) compound for cryosection. The spheroids were sectioned into 10 μM thick slices using a Leica (Buffalo Grove, IL, USA) cryostat platform and then attached onto Leica Apex high adhesive glass slides. A standard immunostaining protocol was used to visualize pluripotency markers for the hiPSC spheroids. Slides were rinsed with 1×PBS to remove OCT and then incubated with 0.1 % Triton X-100 (Sigma-Aldrich) to permeabilize the cells. Non-specific binding was blocked using a 3% BSA solution in 1× PBS for 1 h at room temperature. Afterwards, samples were incubated with primary antibodies at 4 °C overnight (1:500 dilution). The primary antibodies were OCT-4, SSEA-4, SOX2, NANOG. Afterwards, the samples were rinsed with 1×PBS and incubated with the associated secondary antibodies (goat anti-rabbit IgG Alexa Fluor™ 488 and goat anti-mouse IgG Alexa Fluor™ 568 (A-11004, ThermoFisher Scientific), 1:1000 dilution) in 1×PBS for 1 hour at room temperature. The samples were then rinsed with 1×PBS and stained for 10 minutes with 1 μg/mL 4′,6-diamidino-2-phenylindole (DAPI, Sigma-Aldrich) to visualize the nuclei. Samples were imaged with a Zeiss LSM 710 microscope.

Cardiac Differentiation of 3D hiPSC Spheroids

To further analyze the pluripotency difference between 3D hiPSC spheroids from conventional 3D culture (with RI and Versene detachment) and crMPD, cardiac differentiation of the spheroids was studied by using a commercially available PSC Cardiomyocyte Differentiation Kit (ThermoFisher Scientific), per the manufacturer's instructions. Briefly, the DF19-9-11T.H hiPSC spheroids made by either Versene detachment with 10 μM RI supplementation or cold treatment on 400 μm Matrigel island in crMPD with 0 μM RI supplementation were collected after two days of 3D suspension culture. The hiPSC spheroids were transferred to cardiomyocyte differentiation medium A and cultured for 2 days. Then, the spheroids were transferred to cardiomyocyte differentiation medium B and cultured for 2 days. The resultant cardiac spheroids were transferred to cardiomyocyte maintenance medium and cultured for 11 days. Medium were changed every other day.

### Statistical Analysis

All graphing and statistical analyses were performed using GraphPad Prism 8 (GraphPad Software, San Diego, CA, USA). Data are reported as the mean±standard deviation from at least three independent experiments. For comparison between groups, one-way analysis of variance (ANOVA) followed by Tukey post hoc correction, and two-tailed unpaired t-test and two-tailed paired t-test using the parametric test and assuming equal variance, were performed. Differences were considered statistically significant when the p value was less than 0.05. *p<0.05, **p<0.01, ***p<0.001, ****p<0.0001, and ns: not significant.

### Supporting Information

Supporting Information is available from the Wiley Online Library or from the Applicant.

As discussed, the instant invention provides an innovative solution to a current problem: generating sufficient numbers of high-quality and high-yield substantially spheroidal or spheroidal stem cells, i.e., under 3D culture, without the usage of RI or other inhibitors. As such, under 3D culture without RI, the stem cell colonies can quickly self-assemble into homogeneous hiPSC spheroids with high viability, yield, and pluripotency.

While the present invention has been illustrated by the description of the embodiments thereof, and while the embodiments have been described in detail, it is not the intention of the applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the breadth or scope of the applicant's concept. Furthermore, although the present invention has been described in connection with a number of exemplary embodiments and implementations, the present invention is not so limited but rather covers various modifications and equivalent arrangements, which fall within the purview of the appended claims.

What claimed is:

1. A method of stem cell generation comprising:
applying a cold-responsive layer onto a surface;
applying a cell-attachment micropattern onto said cold-responsive layer;
growing stem cells within said cell-attachment micropattern; and
cooling said stem cells on said cold-responsive layer,
whereby said stem cells disengage or detach from said cold-responsive layer without the use of chemical agents.

2. The method of stem cell generation according to claim 1, wherein said stem cells are substantially spheroidal stem cells cultured in 3D to form spheroids.

3. The method of stem cell generation according to claim 1, wherein said stem cells are selected from the group consisting of human induced pluripotent stem cells, embryonic stem cells, adult stem cells, somatic cells, cardiomyocytes, neurons, hepatocytes and combinations thereof.

4. The method of stem cell generation according to claim 1, wherein said cold-responsive layer is a cold-responsive layer spin-coated or spray-coated onto said surface.

5. The method of stem cell generation according to claim 4, wherein said cold-responsive layer is a poly(N-isopropyl acrylamide-butyl acrylate) compound.

6. The method of spheroidal stem cell generation according to claim 4, wherein said cold-responsive layer has a lower critical solution temperature of about 0 to about 12 °C, about 2 to about 8 °C, or about 4 to about 8 °C.

7. The method of stem cell generation according to claim 1, further comprising, prior to the step of applying a cell-attachment micropattern, the step of:
forming a stamp device having a micropattern applicator;
coating said micropattern applicator on said stamp device with a cell adhesion material; and
biostamping, using said micropattern applicator of said stamp device, said cell adhesion material onto said cold-responsive layer, forming said cell-attachment micropattern across said surface comprising respective islands of cell adhesion material.

8. The method of stem cell generation according to claim 7, wherein said stamp device and said micropattern applicator are made of polydimethylsiloane 9. The method of stem cell generation according to claim 7, wherein said cell adhesion material is an extracellular matrix selected from the group consisting of Matrigel, collagen, vitronectin, laminin, gelatin and combinations thereof.

10. The method of stem cell generation according to claim 7, wherein a respective one of said islands of cell adhesion material on said surface is about 10 μm to about 1,000 μm in diameter.

11. The method of stem cell generation according to claim 1, wherein said step of cooling said stem cells on said cold-responsive layer comprises cooling to about 0 degrees Centigrade.

12. The method of stem cell generation according to claim 11, wherein said step of cooling said stem cells on said cold-responsive layer comprises the step of placing said stem cells on said cold-responsive layer in a cold environment selected from the group consisting of: on ice, on a cold surface, in cold air, inside a refrigerator or other icy environment.

13. A device for stem cell generation comprising:
a cold-responsive polymer coated on a surface of said device;
a micropattern of cell-attachment material applied onto said cold-responsive polymer, said micropattern comprising a plurality of spaced apart cell attachment areas along said surface,
whereby stem cells growing on said plurality of cell attachment areas are detachable by cooling said device without the use of chemical agents.

14. The device according to claim 13, wherein said device is a dish, a Petri dish, a flask or other container.

15. The device according to claim 13, wherein said cold-responsive polymer is spin-coated or spray-coated onto said surface of said device.

16. The device according to claim 14, wherein said cold-responsive layer is a poly(N-isopropyl acrylamide-butyl acrylate) compound.

17. The device according to claim 13, wherein said cold-responsive layer has a lower critical solution temperature of about 0 to about 12 °C, about 2 to about 8 °C, or about 4 to about 8 °C.

18. The device according to claim 13, wherein said cell-attachment material placed on said device in a micropattern is an extracellular matrix selected from the group consisting of Matrigel, collagen, vitronectin, laminin, gelatin and combinations thereof.

19. The device according to claim 13, wherein a respective one of plurality of spaced apart cell attachment areas along said surface is about 10 μm to about 1,000 μm in diameter.

20. The device according to claim 13, wherein said device is capable of generating substantially spheroidal stem cells selected from the group consisting of human induced pluripotent stem cells, embryonic stem cells, adult stem cells, somatic cells, cardiomyocytes, neurons, hepatocytes and combinations thereof.
